SEQUENCE LISTING (1 sequence)

SEQ ID NO: 1 - DNA, 25 bp, Artificial Sequence
  random sequence
accgtttaaaggtttccccgtcgta

(19) United States
(12) Patent Application Publication
(10) Pub. No.: US 2019/0277829 A1
(43) Pub. Date: Sep. 12, 2019

(54) HYBRID NANOPORES WITH ANNULAR DNA NANOSTRUCTURES

(71) Applicant: Quantapore, Inc., Menlo Park, CA (US)

(72) Inventors: Jan F. SIMONS, Menlo Park, CA (US); Martin HUBER, Menlo Park, CA (US)

(73) Assignee: Quantapore, Inc., Menlo Park, CA (US)

(21) Appl. No.: 16/347,165

(22) PCT Filed: Nov. 7, 2017
(86) PCT No.: PCT/US2017/060439

§ 371 Date: May 2, 2019

Related U.S. Application Data
(60) Provisional application No. 62/428,322, filed on Nov. 30, 2016.

(51) Int. Cl. G01N 33/487; C12Q 1/06; G01N 27/447

(52) U.S. Cl. CPC G01N 33/48721; B82Y 5/00; G01N 27/447; C12Q 1/06

(57) ABSTRACT

The invention is directed to articles of manufacture for constraining movement of molecules, such as polynucleotides, and methods of using the same. In some embodiments, article of manufacture of the invention comprise (i) a solid state membrane having at least one aperture extending therethrough from a first side to a second side; (ii) an annular DNA sheet having a central opening disposed on the first side of the solid state membrane such that the annular DNA sheet spans an aperture and the central opening is aligned with the aperture to provide fluid communication between the first side and the second side of the solid state membrane through the aperture; and (iii) a protein nanopore immobilized in the central opening of the annular DNA sheet spanning the aperture. Uses of such articles of manufacture include determining sequences of nucleic acids.

### CROSS-REFERENCE TO RELATED APPLICATIONS

This application claims benefit of priority to U.S. Provisional Application No. 62/428,322, filed on Nov. 30, 2016, the content of which is incorporated herein by reference in its entirety.

### BACKGROUND

A challenge of nanopore-based technologies has been the reliable construction of robust nanopores having bores in the sub-10-nanometer range with minimal variance. Current solid state fabrication techniques can reliably produce solid state membranes with nanopores having bore diameters of a few tens of nanometers. Attempts to progress beyond this limit have been made by fabricating so-called hybrid nanopores that consist of a solid state membrane with one or more apertures, or holes, that have protein nanopores inserted into them, for example, by coating the membrane with a lipid bilayer that spans the apertures. Biological protein nanopores have very precise bores in the sub-10 nanometer range. Unfortunately, however, such hybrids have been technically difficult to make and the end products have not been robust.

Since the development of several convenient DNA synthesis and manipulation technologies, techniques have come available for using DNA as a nanostructural material, e.g. Seeman, Chemistry and Biology, 10: 1151-1159 (2003), which relies on a process sometimes referred to as “DNA origami.” Using such technology, attempts have been made to produce DNA nanopores, e.g. Wei et al, Angew. Chem. Int. Ed., 51: 4864-4867 (2012); however, such DNA nanopores, at best, are early stage and lack many favorable properties of protein nanopores, such as, precise sub-10 nanometer bores, suppression of fluorescently labeled DNA translocating through the bore, and the like.

Nanopore-based technologies, such as those used in single molecule analysis, would be advanced by the availability of routinely made components, or articles of manufacture, comprising robust nanopores having bores in the sub-10 nanometer range.

### SUMMARY OF THE INVENTION

The present invention is directed to articles of manufacture comprising hybrid nanopores having bore diameters in the sub-10 nanometer range for use in microfluidic and/or nanofluidic devices and methods of making the same; in particular, the invention includes methods and systems using such hybrid nanopores for determining nucleotide sequences of nucleic acids.

In one aspect, the invention includes articles of manufacture for constraining movement of molecules which comprise the following elements: a solid state membrane having at least one aperture extending therethrough from a first side to a second side; an annular DNA sheet having a central opening disposed on the first side of the solid state membrane such that the annular DNA sheet spans an aperture and the central opening is aligned with the aperture to provide fluid communication between the first side and the second side of the solid state membrane through the aperture; and a protein nanopore immobilized in the central opening of the annular DNA sheet spanning the aperture.

The present invention is exemplified in a number of implementations and applications, some of which are summarized below and throughout the specification.

### BRIEF DESCRIPTION OF THE DRAWINGS

FIGS. 1A-1H illustrate embodiments of the invention.

FIGS. 2A-2C illustrate embodiments of the invention for analyzing nucleic acids, which include quenching agents in a trans chamber, a cis chamber and in both cis and trans chambers.

FIG. 3 illustrates an embodiment of the invention using a protein nanopore and epi-illumination with a metal layer on the nanopore array to reduce background or with TIR and FRET excitation.

FIG. 4 illustrates the basic components of a confocal epi-illumination system.

FIG. 5 illustrates elements of a TIRF system for excitation of optical labels in or near a nanopore array without FRET signal generation.

FIGS. 6A-6C illustrate embodiments employing two and three fluorescent labels.

### DETAILED DESCRIPTION OF THE INVENTION

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention. For example, particular nanopore types and numbers, particular labels, FRET pairs, detection schemes, fabrication approaches of the invention are shown for purposes of illustration. It should be appreciated, however, that the disclosure is not intended to be limiting in this respect, as other types of nanopores, arrays of nanopores, and other fabrication technologies may be utilized to implement various aspects of the systems discussed herein. Guidance for aspects of the invention is found in many available references and treatises well known to those with ordinary skill in the art, including, for example, Cao, Nanostructures & Nanomaterials (Imperial College Press, 2004); Levinson, Principles of Lithography, Second Edition (SPIE Press, 2005); Doering and Nishi, Editors, Handbook of Semiconductor Manufacturing Technology, Second Edition (CRC Press, 2007); Sawyer et al, Electrochemistry for Chemists, 2^{nd} edition (Wiley Interscience, 1995); Bard and Faulkner, Electrochemical Methods: Fundamentals and Applications, 2^{nd} edition (Wiley, 2000); Lakowicz, Principles of Fluorescence Spectroscopy, 3^{rd} edition (Springer, 2006); Hermanson, Bioconjugate Techniques, Second Edition (Academic Press, 2008); and the like, which relevant parts are hereby incorporated by reference.

In one aspect, the invention is directed to articles of manufacture, or products, comprising hybrid nanopores having solid state, protein and nucleic acid components, and the use of such products in molecular analysis, such as, detecting particular molecular analytes, single molecule analysis, nucleic acid sequencing, and the like. In some embodiments, such components comprise a solid state membrane having at least one aperture, an annular DNA sheet covering, or spanning, the aperture and having a central opening, and a protein nanopore immobilized in the central opening so that there is fluid communication across the solid state membrane through the immobilized protein nanopore. In some embodiments, articles of manufacture of the invention comprise a solid state membrane comprising an array of a plurality of apertures, wherein substantially every aperture has disposed thereon an annular DNA sheet having a central opening and wherein substantially every central opening has immobilized therein a protein nanopore. In some embodiments, articles of the invention may be used to constrain the movement of molecules, e.g. to constrain them to move in a single file manner, which, for example, may facilitate their detection or identification. In some embodiments, articles of the invention may be used to constrain the movement of electrically charged molecules or molecules that may be rendered electrically charged by selection of reaction or operating conditions, e.g. pH, ionic composition and concentration, or the like. Exemplary molecules whose movement may be constrained by articles of the invention include, but are not limited to, ionic polymers, charged biopolymers, polynucleotides, proteins, or the like. In particular, such exemplary polynucleotides include DNA or RNA. In some embodiments, such exemplary polynucleotides comprise single stranded DNA. In some embodiments, such exemplary polynucleotides comprise double stranded DNA.

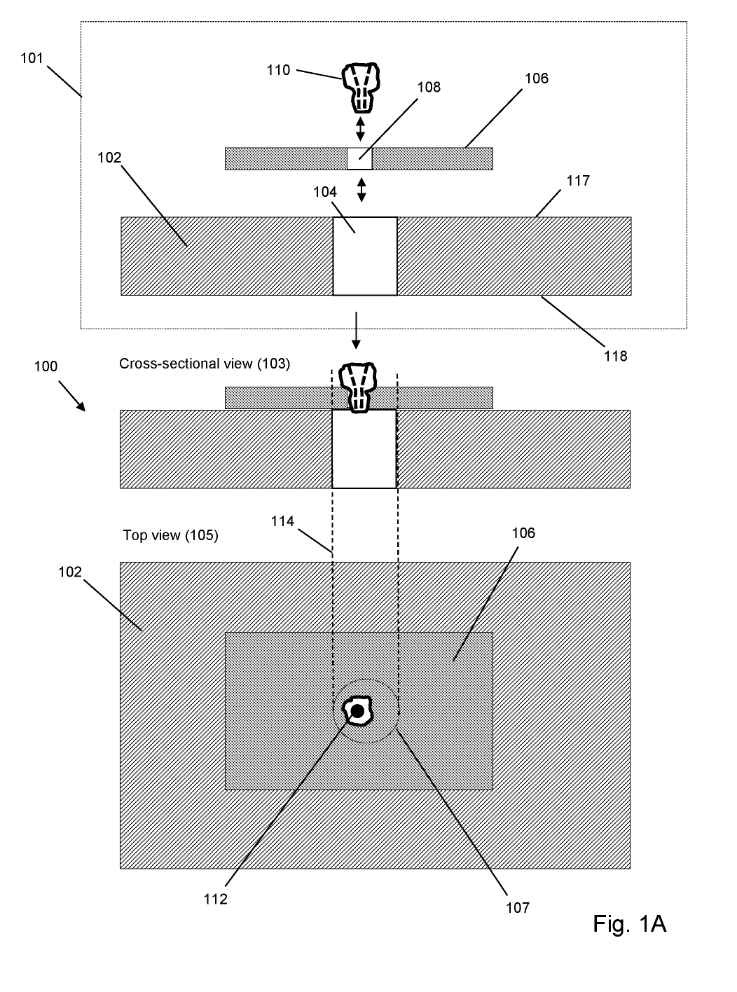

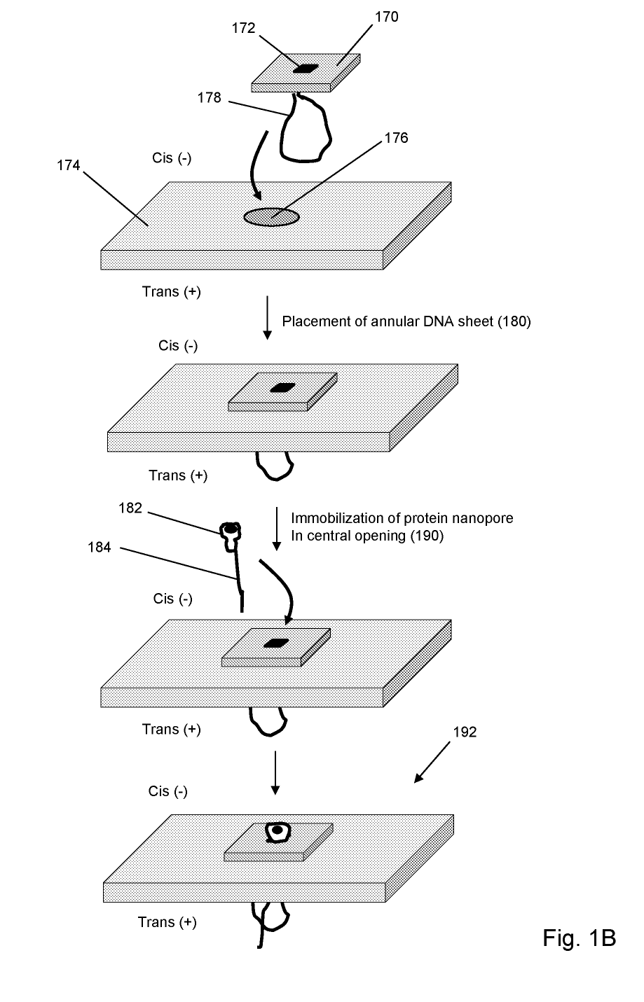

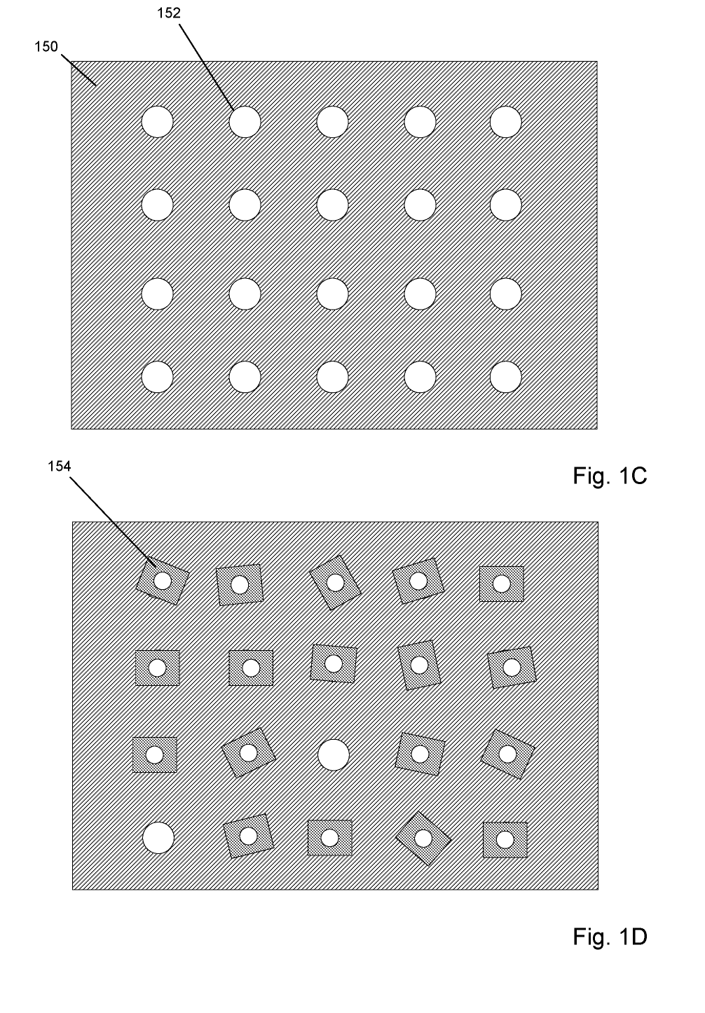

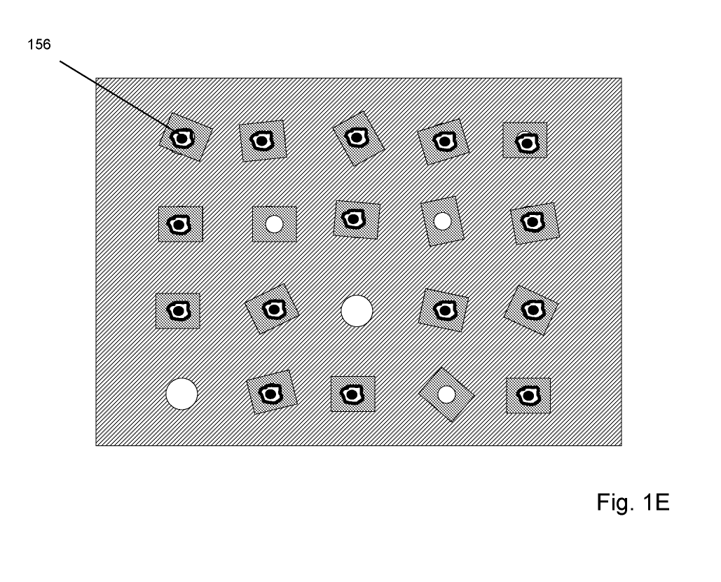

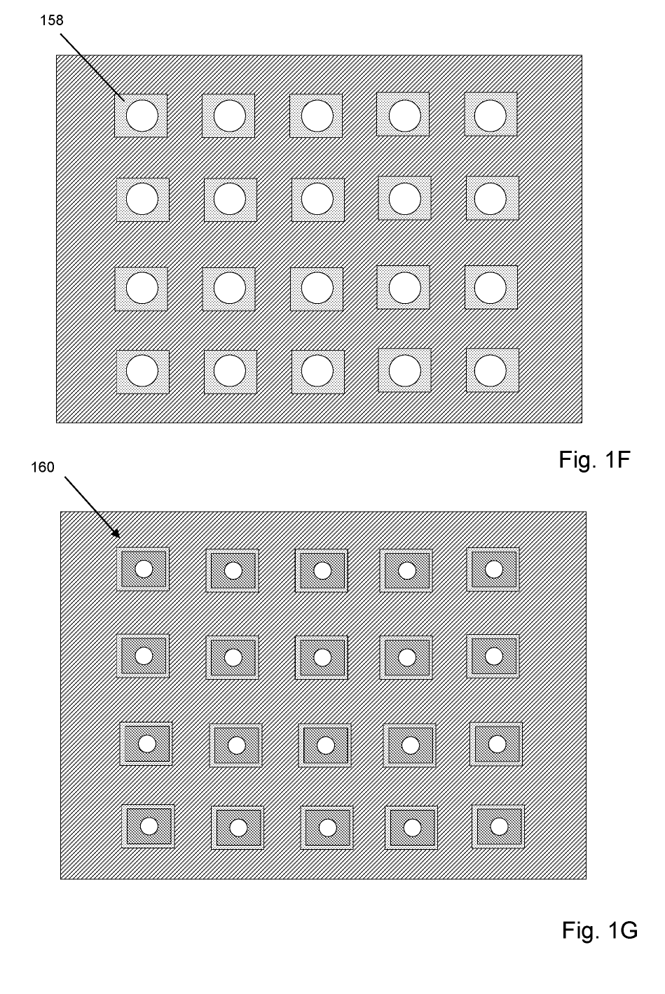

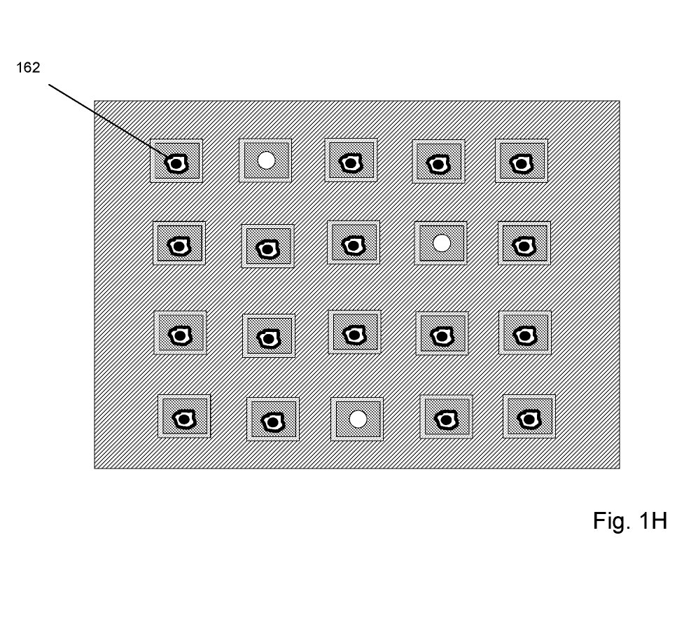

An exemplary embodiment of the invention is illustrated in FIG. 1A. Panel (101) shows three elements of an article of the invention in exploded view. Portion of solid state membrane (102) is shown with aperture (104) over which annular DNA sheet (106) having central opening (108) is positioned. As used herein, the side or surface of solid state membrane (102) on which annular DNA sheet (106) is positioned is sometimes referred to as the first side (117) of solid state membrane (102) and the opposite side is sometimes referred to as the second side (118) of solid state membrane (102). In some embodiments, first side (117) forms part of a cis chamber and second side (118) forms part of a trans chamber. Protein nanopore (110) is immobilized in central opening (108) to give an assembled article of the invention (100), shown below panel (101) in cross-sectional view (103) and top view (105). Annular DNA sheet (106) is positioned on solid state membrane (102) relative to aperture (104) so that central opening (108) and bore (112) of protein nanopore (110) are within the cross-sectional area of aperture (104), as illustrated by dashed lines (114) relating aperture (104) in cross-sectional view (103) to its position which is hidden in top view (105). Position of hidden aperture (104) is indicated additionally by dashed circle (107). Functionally, the positioning is such that there is fluid communication between first side (117) and second side (118) of solid state membrane (102) through bore (112) of protein nanopore (110).

In some embodiments, such protein nanopores have a structure identical to, or similar to, α-hemolysin in that it comprises a barrel, or bore, along an axis and at one end has a “cap” structure and at the other end has a “stem” structure (using the terminology from Song et al, Science, 274: 1859-1866 (1996)). In some embodiments using such protein nanopores, insertion into a central opening of an annular DNA sheet results in the protein nanopore being oriented so that its cap structure is exposed to the cis chamber and its stem structure is exposed to the trans chamber.

Components of article (100) may be stably integrated by physical conditions or by bonding using a variety of bonding agents. Bonding of protein nanopore (110) to annular DNA sheet (106) at central opening (108) may include, but is not limited to, attaching hydrophobic groups to exterior residues of protein nanopore (110) and to DNA components of annular DNA sheet (106) at central opening (108), so that the respective hydrophobic groups may interact to stabilize an inserted protein nanopore. Such hydrophobic groups may include cholesterols, aliphatic groups, porphyrin moieties, and the like, such as disclosed by Burns et al, Angew. Chem. Int. Ed., 52: 12069-12072 (2013); Gryaznov, U.S. Pat. No. 5,571,903; or the like, which references are incorporated herein by reference. An embodiment for assembling and maintaining article (100) in a stably integrated state by physical conditions is illustrated in FIG. 1B.

In some embodiments, protein nanopore (110) and annular DNA sheet (106) may be combined first to form a first precursor product comprising protein nanopore (110) stably inserted into, or immobilized in, central opening (108) of annular DNA sheet, after which the first precursor product may be combined with, and positioned on, solid state membrane (102) to form a final article of the invention (100). In other embodiments, annular DNA sheet (106) and solid state membrane (102) may be combined first to form a second precursor product comprising annular DNA sheet positioned on, and stably attached to, solid state membrane (102), after which the second precursor product may be combined with protein nanopore (110) to form article (100) of the invention.

FIG. 1B illustrates the stepwise assembly and integration of article (100) by electrophoretically guiding components to apertures (176) of solid state membrane (174). The electric field used to assemble the component is also used to hold them stably in place. In the illustrated embodiment, annular DNA sheet (170) is assembled using the technique of Wei et al, Angew. Chem. Int. Ed., 51: 4864-4867 (2012) (including supplemental materials), which are incorporated herein by reference. Briefly, a scaffold DNA is used to assemble a multitude of “staple” DNAs. In the particular construction of annular DNA sheet (170), a portion (178) of a scaffold DNA hangs free from a location near central opening (172) so that under the influence of an electric field it may be drawn to, captured and held in place (180) at aperture (176). Because free loop (178) and annular DNA sheet (170) are negatively charged under the reaction conditions, annular DNA sheet (170) may be place in a cis chamber and drawn to a trans chamber on the opposite side of solid state membrane (174). In an analogous manner, polynucleotide (184) may be attached to protein nanopore (182) to form a conjugate that under an electric field is drawn to and captured by (190) central opening (172) to form a final article (192). In some embodiments, so long as an electrical field is maintained across solid state membrane (174), the article will remain stably integrated.

As illustrated in FIGS. 1C-1E, the above operations may be performed to form an array of hybrid nanopores. FIG. 1C shows solid state membrane (150) with a plurality of apertures (152). In step one of the process described in FIG. 1B, annular DNA sheets (154) may be guided to apertures of solid state membrane (150), shown in FIG. 1D. As also noted in FIG. 1D, the orientations of rectangular annular DNA sheets (154) are essentially random, and as also noted, not all apertures may have annular DNA sheets. In step two of the process described in FIG. 1B, protein nanopores (156) are guided to central openings of annular DNA sheets covering apertures, as shown in FIG. 1E. In some embodiments, the plurality of apertures may be at least 2; in other embodiments, the plurality may be in the range of from 2 to 10,000; in other embodiments, the plurality may be in the range of from 16 to 1000, or in the range of from 16 to 10,000.

In some embodiments, articles of the invention are formed by bonding annular DNA sheets to solid state membranes using various bonding agents and methods, such as those disclosed by Gopinath et al (cited below). Without specific physical guidance mechanisms as described above, placement of DNA structures on a solid state surface would be random without specially prepared sites (referred to herein as “landing sites”) which preferentially capture and orient the DNA structures. In accordance with one embodiment of the invention, solid state membrane (159) is modified to include landing sites (158) adjacent to apertures (157) which are configured to accept an annular DNA sheet in a desired orientation. Typically landing sites are prepared by changing the surface chemistry in the landing site so that functionalities may be attached that preferentially bind complementary functionalities on an annular DNA sheet (i.e. positioning agents or functionalities) or that permit chemical crosslinking with complementary functionalities on an annular DNA sheet (or both). As illustrated in FIG. 1G, annular DNA sheets may be positioned (160) on landing sites (161) in a desired orientation, after which protein nanopores (162) may be immobilized in central opening as described above.

### Construction of Annular DNA Sheets and their Deposition onto Lithographically Patterned Surfaces

Annular DNA sheets of the invention are planar DNA nanostructures that serve as adaptors between one or more protein nanopores and an aperture of a solid state membrane. Such DNA nanostructures may be constructed in a variety of ways described in references cited below. Construction approaches and terminology are reviewed by Kuzuya et al, Nanoscale, 2: 310-322 (2010), which is incorporated herein by reference. The particular geometry of an annular DNA sheet may vary widely; however, in some embodiments, its shape is planar with first and second surface areas of a magnitude and geometry such that it is capable of covering an aperture and with at least one central opening configured to immobilize a protein nanopore. In some embodiments, the thickness of an annular DNA sheet may vary from the width of a DNA double helix (2-3 nanometer) to tens of nanometers (e.g. 10-20 nanometers) depending on the DNA components and substructures employed. The surface area of an annular DNA sheet depends on the diameter of the apertures to be covered, the degree of overlap desired between the annular DNA sheet and the solid state membrane, the nature of the complementary functionalities and positioning agents employed, and the like. In some embodiments, an annular DNA sheet has a surface area and geometry to cover an aperture of an approximate diameter of 100 nm, or 50 nm, or 20 nm, or 10 nm. In some embodiments, an annular DNA sheet is rectilinear with a width in the range of from 10 to 80 nm and a length in the range of 10 to 150 nm; and in further embodiments, such rectilinear sheet has a single central opening at its center. In some embodiments, the geometry of an annular DNA sheet may be convex, rectilinear, square, triangular, hexagonal, circular, or oval. An annular DNA sheet usually has a single central opening; however, in some embodiments, an annular DNA sheet may have a plurality of central openings, such as, 2 to 6 central openings, or 2 to 4 central openings, or 2 central openings. The cross-sectional area and geometry of central openings may vary depending on the kind of protein nanopore immobilized therein and whether an immobilizing agent is used to increase the stability of the immobilization, that is, the protein nanopore-annular DNA sheet complex. Exemplary immobilizing agent include, but are not limited to, cross-linkers that form covalent or non-covalent bonds between the protein nanopore and the annular DNA sheet. Exemplary covalent linkages include those formed by conventional linking agents, such as linkers that connect amine groups to thiols, or amine groups to carboxyl groups, or amine groups to amine groups, or amine groups to aldehyde groups. Covalent linkages may also be formed by click chemistries.

An extensive literature is available to those of ordinary skill in the art for design and assembly of DNA nanostructures, such as those called for in the present invention, including the following references that are incorporated herein by reference: U.S. Pat. Nos. 7,842,793; 8,501,923; 9340416; 9371155; Rothemund et al, Nature, 440: 297-302 (2006) including supplemental material; Douglas et al, Nature, 459(7245): 414-418 (2009) including supplemental material; Douglas et al, Nucleic Acids Research, 37(15): 5001-5006 (2009); Castro et al, Nature Methods, 8(3): 221-229 (2011) including supplemental materials; and the like.

Likewise, an extensive literature is available describing methods and materials for positioning and bonding DNA nanostructures on lithographically patterned surfaces, including the following references that are incorporated herein by reference: Kershner et al, Nature Nanotechnology, 4(9): 557-561 (2009) including supplemental materials; Gopinath et al, ACS Nano, 8(12): 12030-12040 (2014) including supplemental material; Gopinath et al, Nature, 535: 401-405 (2016) including supplemental materials; and the like. In some embodiments, positioning is accomplished by providing DNA components of the annular DNA sheet with nucleotides having first reactive moieties, such as primary amines, and a lithographically prepared landing site derivatized with second reactive moieties, such as carboxyl groups, e.g. Gopinath et al (2016, cited above) on the solid state membrane. The annular DNA sheets are positioned on the landing sites by formation of a non-covalent Mg⁺² salt bridge between the negatively charged annular DNA sheets and negatively charged silanol groups at the landing site. After incubation to allow the annular DNA sheets on landing sites to reach a minimal free energy state corresponding to maximal overlap (and therefore alignment of the annular DNA sheets with their respective landing sites), the silanol groups are converted into carboxyl groups and then are cross-linked with the free amines on the annular DNA sheet via a crosslinking agent, such as, 1-Ethyl-3-(3-dimethylaminopropyl)carbodiimide (EDC).

In other embodiments, other pairs of reactive moieties and cross-linking agents may be used, such as thiol groups, amine groups and a cross-linking agent such as succinimidyl trans-4-(maleimidylmethyl)cyclohexane-1-carboxylate (SMCC), or the like, e.g. see Hermanson (cited above) for further examples.

In other embodiments, the reactive moiety on the annular DNA sheets may be a thiol group that forms a covalent bond with a gold surface, i.e. the SiN membrane with the synthetic nanopore is covered with a thin gold film. The thiol groups of the annular DNA sheet will form a covalent bond with the gold surface once placed over the synthetic nanopore.

In still other embodiments, landing sites and annular DNA sheets may be derivatized with complementary DNA strands (referred to herein as “docking strands”) that “dock” an annular DNA sheet at a landing site by forming duplexes. As above, such strands may be distributed on the annular DNA sheet and landing site so that, after an incubation period (which may include raising and lowering the temperature for re-annealing), a minimal free energy state is reached corresponding to a desired alignment of the annular DNA sheet and the landing site. After such docking, the docking strands forming duplexes may be cross-linked using conventional reagents, e.g. photoactivated psoralen. As used herein, compounds, moieties, chemical groups, and the like, used to align or position an annular DNA sheet with a landing site are sometimes referred to herein as positioning agents. As used herein, compounds, moieties, chemical groups, and the like, used to stably fix an annular DNA sheet to a landing site are sometimes referred to herein as bonding agents. In some embodiments, bonding agents form, or assist in the formation, of covalent linkages between a solid state membrane at a landing site and an annular DNA sheet.

Annular DNA sheets constructed by conventional DNA origami techniques are highly modular, so that a wide variety of different positioning agents and/or bonding agents may be used without the need for a major re-design of polynucleotide components. A wide variety of immobilizing agents may be used with protein nanopores and/or a wide variety of positioning agents or bonding agents may be used by only modifying nucleotides of polynucleotide components forming the surface of a central opening or directly aligned with landing sites, respectively.

In some embodiments, articles of the invention may be assembled in the following steps: (a) providing a solid state membrane having at least one aperture extending therethrough from a first side to a second side; (b) positioning an annular DNA sheet having a central opening on the first side of the solid state membrane such that the annular DNA sheet spans an aperture and the central opening is aligned with the aperture to provide fluid communication between the first side and the second side of the solid state membrane through the aperture; and (c) immobilizing a protein nanopore in the central opening of the annular DNA sheet spanning the aperture.

In other embodiments, articles of the invention may be assembled in the following steps: (a) providing a solid state membrane having at least one aperture extending therethrough from a first side to a second side and having in proximity to each of the apertures on the first side a defined surface region specific to the aperture; (b) positioning an annular DNA sheet having a central opening on the first side of the solid state membrane such that (i) the annular DNA sheet spans an aperture and overlaps the defined surface region, and (ii) the central opening is aligned with the aperture to provide fluid communication between the first side and the second side of the solid state membrane through the aperture; and (c) immobilizing a protein nanopore in the central opening of the annular DNA sheet spanning the aperture. In some embodiments, the defined surface region has a first shape and the annular DNA sheet has a second shape. In some embodiments the first and second shapes are complementary. In other embodiments the first and second shapes are the same and the areas of the defined surface region and the annular DNA sheet are the same. In some embodiments, the defined surface region provides a landing site for an annular DNA sheet; that is, the defined surface region provides a contact surface for an annular DNA sheet which has a minimal free energy alignment that corresponds to the correct positioning of the central opening with respect to the aperture. In some embodiments, the defined surface regions are prepared using conventional micromachining techniques, such as defining shapes, areas and coatings using conventional lithographic masking and etching techniques.

### Solid State Membranes, Apertures and Nanopores

Important features of nanopores include constraining polynucleotide analytes, such as labeled polynucleotides so that their monomers pass through a signal generation region (or equivalently, an excitation zone, or detection zone, or the like) in sequence. That is, a nanopore contrains the movement of a polynucleotide analyte, such as a polynucleotide, so that nucleotides pass through a detection zone (or excitation region) in single file. In some embodiments, additional functions of nanopores include (i) passing single stranded nucleic acids while not passing double stranded nucleic acids, or equivalently bulky molecules and/or (ii) constraining fluorescent labels on nucleotides so that fluorescent signal generation is suppressed or directed so that it is not collected.

In some embodiments, nanopores used in connection with the methods and devices of the invention are provided in the form of arrays, such as an array of clusters of nanopores, which may be disposed regularly on a planar surface. In some embodiments, clusters are each in a separate resolution limited area so that optical signals from nanopores of different clusters are distinguishable by the optical detection system employed, but optical signals from nanopores within the same cluster cannot necessarily be assigned to a specific nanopore within such cluster by the optical detection system employed.

Solid state membranes with apertures (sometime referred to as “solid state nanopores”) may be fabricated in a variety of materials including but not limited to, silicon nitride (Si₃N₄), silicon dioxide (SiO₂), and the like. The fabrication and operation of solid state nanopores for analytical applications, such as DNA sequencing, are disclosed in the following exemplary references that are incorporated by reference: Ling, U.S. Pat. No. 7,678,562; Hu et al, U.S. Pat. No. 7,397,232; Golovchenko et al, U.S. Pat. No. 6,464,842; Chu et al, U.S. Pat. No. 5,798,042; Sauer et al, U.S. Pat. No. 7,001,792; Su et al, U.S. Pat. No. 7,744,816; Church et al, U.S. Pat. No. 5,795,782; Bayley et al, U.S. Pat. No. 6,426,231; Akeson et al, U.S. Pat. No. 7,189,503; Bayley et al, U.S. Pat. No. 6,916,665; Akeson et al, U.S. Pat. No. 6,267,872; Meller et al, U.S. patent publication 2009/0029477; Howorka et al, International patent publication WO2009/007743; Brown et al, International patent publication WO2011/067559; Meller et al, International patent publication WO2009/020682; Polonsky et al, International patent publication WO2008/092760; Van der Zaag et al, International patent publication WO2010/007537; Yan et al, Nano Letters, 5(6): 1129-1134 (2005); Iqbal et al, Nature Nanotechnology, 2: 243-248 (2007); Wanunu et al, Nano Letters, 7(6): 1580-1585 (2007); Dekker, Nature Nanotechnology, 2: 209-215 (2007); Storm et al, Nature Materials, 2: 537-540 (2003); Wu et al, Electrophoresis, 29(13): 2754-2759 (2008); Nakane et al, Electrophoresis, 23: 2592-2601 (2002); Zhe et al, J. Micromech. Microeng., 17: 304-313 (2007); Henriquez et al, The Analyst, 129: 478-482 (2004); Jagtiani et al, J. Micromech. Microeng., 16: 1530-1539 (2006); Nakane et al, J. Phys. Condens. Matter, 15 R1365-R1393 (2003); DeBlois et al, Rev. Sci. Instruments, 41(7): 909-916 (1970); Clarke et al, Nature Nanotechnology, 4(4): 265-270 (2009); Bayley et al, U.S. patent publication 2003/0215881; and the like.

In some embodiments, the invention comprises nanopore arrays with one or more light-blocking layers, that is, one or more opaque layers. Typically nanopore arrays are fabricated in thin sheets of material, such as, silicon, silicon nitride, silicon oxide, aluminum oxide, or the like, which readily transmit light, particularly at the thicknesses used, e.g. less than 50-100 nm. For electrical detection of analytes this is not a problem. However, in optically-based detection of labeled molecules translocating nanopores, light transmitted through an array invariably excites materials outside of intended reaction sites, thus generates optical noise, for example, from nonspecific background fluorescence, fluorescence from labels of molecules that have not yet entered a nanopore, or the like. In one aspect, the invention addresses this problem by providing nanopore arrays with one or more light-blocking layers that reflect and/or absorb light from an excitation beam, thereby reducing background noise for optical signals generated at intended reaction sites associated with nanopores of an array. In some embodiments, this permits optical labels in intended reaction sites to be excited by direct illumination. In some embodiments, an opaque layer may be a metal layer. Such metal layer may comprise Sn, Al, V, Ti, Ni, Mo, Ta, W, Au, Ag or Cu. In some embodiments such metal layer may comprise Al, Au, Ag or Cu. In still other embodiments, such metal layer may comprise aluminum or gold, or may comprise solely aluminum. The thickness of an opaque layer may vary widely and depends on the physical and chemical properties of material composing the layer. In some embodiments, the thickness of an opaque layer may be at least 5 nm, or at least 10 nm, or at least 40 nm. In other embodiments, the thickness of an opaque layer may be in the range of from 5-100 nm; in other embodiments, the thickness of an opaque layer may be in the range of from 10-80 nm. An opaque layer need not block (i.e. reflect or absorb) 100 percent of the light from an excitation beam. In some embodiments, an opaque layer may block at least 10 percent of incident light from an excitation beam; in other embodiments, an opaque layer may block at least 50 percent of incident light from an excitation beam.

Opaque layers or coatings may be fabricated on solid state membranes by a variety of techniques known in the art. Material deposition techniques may be used including chemical vapor deposition, electrodeposition, epitaxy, thermal oxidation, physical vapor deposition, including evaporation and sputtering, casting, and the like. In some embodiments, atomic layer deposition may be used, e.g. U.S. Pat. No. 6,464,842; Wei et al, Small, 6(13): 1406-1414 (2010), which are incorporated by reference.

In some embodiments, a 1-100 nm channel or aperture may be formed through a solid substrate, usually a planar substrate, such as a membrane, through which an analyte, such as single stranded DNA, is induced to translocate. In other embodiments, a 2-50 nm channel or aperture is formed through a substrate; and in still other embodiments, a 2-30 nm, or a 2-20 nm, or a 3-30 nm, or a 3-20 nm, or a 3-10 nm channel or aperture if formed through a substrate.

In some embodiments, methods and devices of the invention comprise a solid phase membrane, such as a SiN membrane, having an array of apertures therethrough providing communication between a first chamber and a second chamber (also sometimes referred to as a “cis chamber” and a “trans chamber”). In some embodiments, diameters of the aperture in such a solid phase membrane may be in the range of 10 to 200 nm, or in the range of 20 to 100 nm. In some embodiments, such solid phase membranes further include protein nanopores inserted into the lipid bilayer in regions where such bilayer spans the apertures on the surface facing the trans chamber. In some embodiments, such protein nanopores are inserted from the cis side of the solid phase membrane using techniques described herein.

### Molecular Analysis Using Articles of the Invention

As mentioned above, articles of the invention may be used to analyze molecules by a variety of approaches including, but not limited to, electrical or optical signatures generated as a molecule of interest passes through the bore of a protein nanopore of the article. Of particular interest is the analysis of single molecules by way of optical signatures they generate as they pass, or translocate, through the bore of a protein nanopore of the article. Such optical signatures may come from an analyte directly or from an optical label attached to the analyte, or both. In some embodiments, analytes detected by devices using an article of the invention include polynucleotides labeled with one of more optical labels, particularly one or more optical labels that generate distinguishable signals that permit nucleotides to which they are attached to be identified. That is, in some embodiments, articles of the invention are used in a device from determining a nucleotide sequence of a polynucleotide.

In some embodiments, a device for implementing the above methods for analyzing polynucleotides (such as single stranded polynucleotides) typically includes a set of electrodes for establishing an electric field across the layered membrane and nanopores. Single stranded nucleic acids are exposed to nanopores by placing them in an electrolyte in a first chamber, which is configured as the “cis” side of the layered membrane by placement of a negative electrode in the chamber. Upon application of an electric field, the negatively single stranded nucleic acids are captured by nanopores and translocated to a second chamber on the other side of the layered membrane, which is configured as the “trans” side of membrane by placement of a positive electrode in the chamber. The speed of translocation depends in part on the ionic strength of the electrolytes in the first and second chambers and the applied voltage across the nanopores. In optically based detection, a translocation speed may be selected by preliminary calibration measurements, for example, using predetermined standards of labeled single stranded nucleic acids that generate signals at different expected rates per nanopore for different voltages. Thus, for DNA sequencing applications, a translocation speed may be selected based on the signal rates from such calibration measurements. Consequently, from such measurements a voltage may be selected that permits, or maximizes, reliable nucleotide identifications, for example, over an array of nanopores. In some embodiments, such calibrations may be made using nucleic acids from the sample of templates being analyzed (instead of, or in addition to, predetermined standard sequences). In some embodiments, such calibrations may be carried out in real time during a sequencing run and the applied voltage may be modified in real time based on such measurements, for example, to maximize the acquisition of nucleotide-specific signals.

### Controlling Translocation Speed of Nucleic Acid Analytes

The role of translocation speed of polynucleotides through nanopores and the need for its control have been appreciated in the field of nanopore technology wherein changes in electric current are use to identify translocating analytes. A wide variety of methods have been used to control translocation speed, which include both methods that can be adjusted in real-time without significant difficulty (e.g. voltage potential across nanopores, temperature, and the like) and methods that can be adjusted during operation only with difficulty (reaction buffer viscosity, presence or absence of charged side chains in the bore of a protein nanopore, ionic composition and concentration of the reaction buffer, velocity-retarding groups attached or hybridized to polynucleotide analytes, molecular motors, and the like), e.g. Bates et al, Biophysical J., 84: 2366-2372 (2003); Carson et al, Nanotechnology, 26(7): 074004 (2015); Yeh et al, Electrophoresis, 33(23): 58-65 (2012); Meller, J. Phys. Cond. Matter, 15: R581-R607 (2003); Luan et al, Nanoscale, 4(4): 1068-1077 (2012); Keyser, J. R. Soc. Interface, 8: 1369-1378 (2011); and the like, which are incorporated herein by reference. In some embodiments, a step or steps are included for active control of translocation speed while a method of the invention is being implemented, e.g. voltage potential, temperature, or the like; in other embodiments, a step or steps are included that determine a translocation speed that is not actively controlled or changed while a method of the invention is being implemented, e.g. reaction buffer viscosity, ionic concentration, and the like. In regard to the latter, in some embodiments, a translocation speed is selected by providing a reaction buffer having a concentration of glycerol, or equivalent reagent, in the range of from 1 to 60 percent.

In regard to the former embodiments (with real-time translocation speed adjustment), a measure of whether one or more than one label is contributing fluorescence to measured signals may be based on the distribution of fluorescence intensity among a plurality of channels over which fluorescence is collected. Typically the plurality of channels include 2, 3, or 4 channels corresponding to the emission bands of the fluorescent labels used. In a measured sample of fluorescence emanating from a region adjacent to a nanopore exit, if only a single label contributes to a measured signal, the relative distribution of signal intensity among the different channels (e.g. 4 channels) could be represented ideally as (1,0,0,0); (0,1,0,0); (0,0,1,0) or (0,0,0,1). On the other hand, if more than one label contributed to a measured fluorescent signal, the relative distributions would include non-zero values in more than one channel, with a worse case being four different labels contributing equally, which would appear as (0.25,0.25,0.25,0.25) in the above representation. A measure which would vary monotonically between a maximum value corresponding to relative intensity distributions (1,0,0,0); (0,1,0,0); (0,0,1,0) or (0,0,0,1) and a minimum value corresponding to a relative intensity distribution of (0.25,0.25,0.25,0.25) may be used for controlling in real-time a translocation speed. For example, an initial translocation speed could be lowered based on the value of such a measure that was near its minimum. Such lowering may be implemented, for example, by lowering a potential voltage across the nanopores by a predetermined amount, after which the measure could be re-calculated. Such steps could be repeated until the process was optimized.

As mentioned above, translocation speeds depend in part on the voltage difference (or electrical field strength) across a nanopore and conditions in the reaction mixture, or buffer, of a first chamber where polynucleotides are exposed to the nanopores (e.g. disposed in a solid phase membrane making up one wall of the first chamber). Polynucleotide capture rates by nanopores depend on concentration of such polynucleotides. In some embodiments, conventional reaction mixture conditions for nanopore sequencing may be employed with the invention (for controlling translocatin speed by varying voltage potential across nanopores), for example, 1M KCl (or equivalent salt, such as NaCl, LiCl, or the like) and a pH buffering system (which, for example, ensures that proteins being used, e.g. protein nanopores, nucleases, or the like, are not denatured). In some embodiments, a pH buffering system may be used to keep the pH substantially constant at a value in the range of 6.8 to 8.8. In some embodiments, a voltage difference across the nanopores may be in the range of from 70 to 200 mV. In other embodiments, a voltage difference across the nanopores may be in the range of from 80 to 150 mV. An appropriate voltage for operation may be selected using conventional measurement techniques. Current (or voltage) across a nanopore may readily be measured using commercially available instruments. A voltage difference may be selected so that translocation speed is within a desired range. In some embodiments, a range of translocation speeds comprises those speeds less than 1000 nucleotides per second. In other embodiments, a range of translocation speeds is from 10 to 800 nucleotides per second; in other embodiments, a range of translocation speeds is from 10 to 600 nucleotides per second; in other embodiments, a range of translocation speeds is from 200 to 800 nucleotides per second; in other embodiments, a range of translocation speeds is from 200 to 500 nucleotides per second. Likewise, other factors affecting translocation speed, e.g. temperature, viscosity, ion concentration, charged side chains in the bore of a protein nanopore, and the like, may be selected to obtain translocation speeds in the ranges cited above.

In some embodiments, a device for implementing the above methods for single stranded nucleic acids typically includes providing a set of electrodes for establishing an electric field across the nanopores (which may comprise an array). Single stranded nucleic acids are exposed to nanopores by placing them in an electrolyte (i.e. reaction buffer) in a first chamber, which is configured as the “cis” side of the layered membrane by placement of a negative electrode in the chamber. Upon application of an electric field, the negatively single stranded nucleic acids are captured by nanopores and translocated to a second chamber on the other side of the layered membrane, which is configured as the “trans” side of membrane by placement of a positive electrode in the chamber. As mentioned above, the speed of translocation depends in part on the ionic strength of the electrolytes in the first and second chambers and the applied voltage across the nanopores. In optically based detection, a translocation speed may be selected by preliminary calibration measurements, for example, using predetermined standards of labeled single stranded nucleic acids that generate signals at different expected rates per nanopore for different voltages. Thus, for DNA sequencing applications, an initial translocation speed may be selected based on the signal rates from such calibration measurements, as well as the measure based on relative signal intensity distribution discussed above. Consequently, from such measurements a voltage may be selected that permits, or maximizes, reliable nucleotide identifications, for example, over an array of nanopores. In some embodiments, such calibrations may be made using nucleic acids from the sample of templates being analyzed (instead of, or in addition to, predetermined standard sequences). In some embodiments, such calibrations may be carried out in real time during a sequencing run and the applied voltage may be modified in real time based on such measurements, for example, to maximize the acquisition of nucleotide-specific signals.

### Embodiments Employing Mutually and Self-Quenching Labels

As mentioned above, in some embodiments, self- and mutually quenching fluorescent labels may be used in addition to quenching agents in order to reduce fluorescent emissions outside of those from labels on nucleotides exiting nanopores. Use of such fluorescent labels is disclosed in U.S. patent publication 2016/0122812, which is incorporated by reference. In some embodiments, monomers are labeled with fluorescent labels that are capable of at least three states while attached to a target polynucleotide: (i) A substantially quenched state wherein fluorescence of an attached fluorescent label is quenched by a fluorescent label on an immediately adjacent monomer; for example, a fluorescent label attached to a polynucleotide in accordance with the invention is substantially quenched when the labeled polynucleotide is free in conventional aqueous solution for studying and manipulating the polynucleotide. (ii) A sterically constrained state wherein a labeled polynucleotide is translocating through a nanopore such that the free-solution movements or alignments of an attached fluorescent label is disrupted or limited so that there is little or no detectable fluorescent signal generated from the fluorescent label. (iii) A transition state wherein a fluorescent label attached to a polynucleotide transitions from the sterically constrained state to the quenched state as the fluorescent label exits the nanopore (during a “transition interval”) while the polynucleotide translocates through the nanopore.

In part, this example is an application of the discovery that during the transition interval a fluorescent label (on an otherwise substantially fully labeled and self-quenched polynucleotide) is capable of generating a detectable fluorescent signal. Without the intention of being limited by any theory underlying this discovery, it is believed that the fluorescent signal generated during the transition interval is due to the presence of a freely rotatable dipole in the fluorescent label emerging from the nanopore, which renders the fluorescent label temporarily capable of generating a fluorescent signal, for example, after direct excitation or via FRET. In both the sterically constrained state as well as the quenched state, the dipoles are limited in their rotational freedom thereby reducing or limiting the number of emitted photons. In some embodiments, the polynucleotide is a polynucleotide, usually a single stranded polynucleotide, such as, DNA or RNA, but especially single stranded DNA. In some embodiments, the invention includes a method for determining a nucleotide sequence of a polynucleotide by recording signals generated by attached fluorescent labels as they exit a nanopore one at a time as a polynucleotide translocates through the nanopore. Upon exit, each attached fluorescent label transitions during a transition interval from a constrained state in the nanopore to a quenched state on the polynucleotide in free solution. In other words, in some embodiments, a step of the method of the invention comprises exciting each fluorescent label as it is transitioning from a constrained state in the nanopore to a quenched state on the polynucleotide in free solution. As mentioned above, during this transition interval or period the fluorescent label is capable of emitting a detectable fluorescent signal indicative of the nucleotide it is attached to.

In some embodiments, the invention includes an application of the discovery that fluorescent labels and nanopores may be selected so that during translocation of a polynucleotide through a nanopore fluorescent labels attached to monomers are forced into a constrained state in which they are incapable (or substantially incapable) of producing a detectable fluorescent signal. In some embodiments, nanopores are selected that have a bore, or lumen, with a diameter in the range of from 1 to 4 nm; in other embodiments, nanopores are selected that have a bore or lumen with a diameter in the range of from 2 to 3 nm. In some embodiments, such bore diameters are provided by a protein nanopore. In some embodiments, such nanopores are used to force fluorescent labels into a constrained state in accordance with the invention, so that whenever a fluorescent label exits a nanopore, it transitions from being substantially incapable of generating a fluorescent signal to being detectable and identifiable by a fluorescent signal it can be induced to emit. Thus, fluorescent labels attached to each of a sequence of monomers of a polynucleotide may be detected in sequence as they suddenly generate a fluorescent signal in a region immediately adjacent to a nanopore exit (a “transition zone” or “transition volume” or “detection zone”). In some embodiments, organic fluorescent dyes are used as fluorescent labels with nanopores of the above diameters. In some embodiments, at least one such organic fluorescent dye is selected from the set consisting of xanthene dyes, rhodamine dyes and cyanine dyes. Some embodiments for determining a monomer sequence of a polynucleotide may be carried out with the following steps: (a) translocating a polynucleotide through a nanopore, wherein monomers of the polynucleotide are labeled with fluorescent labels wherein the nanopore constrains fluorescent labels within its bore into a constrained state such that substantially no detectable fluorescent signal is generated therein; (b) exciting the fluorescent label of each monomer upon exiting the nanopore; (c) measuring a fluorescent signal in a detection zone generated by the exiting fluorescent label to identify the monomer to which the fluorescent label is attached; (d) quenching fluorescent signals from excited fluorescent labels outside of the detection zone, and (d) determining a monomer sequence of the polynucleotide from a sequence of fluorescent signals. In further embodiments, fluorescent labels are acceptors of a FRET pair and one or more donors of the FRET pair are attached to the nanopore within a FRET distance of the exit.

In some embodiments, “substantially quenched” as used above means a fluorescent label generates a fluorescent signal at least thirty percent reduced from a signal generated under the same conditions, but without adjacent mutually quenching labels. In some embodiments, “substantially quenched” as used above means a fluorescent label generates a fluorescent signal at least fifty percent reduced from a signal generated under the same conditions, but without adjacent mutually quenching labels.

In some embodiments, a nucleotide sequence of a target polynucleotide is determined by carrying out four separate reactions in which copies of the target polynucleotide have each of its four different kinds of nucleotide (A, C, G and T) labeled with a single fluorescent label. In a variant of such embodiments, a nucleotide sequence of a target polynucleotide is determined by carrying out four separate reactions in which copies of the target polynucleotide have each of its four different kinds of nucleotide (A, C, G and T) labeled with one fluorescent label while at the same time the other nucleotides on the same target polynucleotide are labeled with a second fluorescent label. For example, if a first fluorescent label is attached to A's of the target polynucleotide in a first reaction, then a second fluorescent label is attached to C's, G's and T's (i.e. to the “not-A” nucleotides) of the target polynucleotides in the first reaction. Likewise, in continuance of the example, in a second reaction, the first label is attached to C's of the target polynucleotide and the second fluorescent label is attached to A's, G's and T's (i.e. to the “not-C” nucleotides) of the target polynucleotide. And so on, for nucleotides G and T.

The same labeling scheme may be expressed in terms of conventional terminology for subsets of nucleotide types; thus, in the above example, in a first reaction, a first fluorescent label is attached to A's and a second fluorescent label is attached to B's; in a second reaction, a first fluorescent label is attached to C's and a second fluorescent label is attached to D's; in a third reaction, a first fluorescent label is attached to G's and a second fluorescent label is attached to H's; and in a fourth reaction, a first fluorescent label is attached to T's and a second fluorescent label is attached to V's.

In some embodiments, a polymer, such as a polynucleotide or peptide, may be labeled with a single fluorescent label attached to a single kind of monomer, for example, every T (or substantially every T) of a polynucleotide is labeled with a fluorescent label, e.g. a cyanine dye. In such embodiments, a collection, or sequence, of fluorescent signals from the polynucleotide may form a signature or fingerprint for the particular polynucleotide. In some such embodiments, such fingerprints may or may not provide enough information for a sequence of monomers to be determined.

In some embodiments, a feature of the invention is the labeling of substantially all monomers of a polynucleotide analyte with fluorescent dyes or labels that are members of a mutually quenching set. The use of the term “substantially all” in reference to labeling polynucleotide analytes is to acknowledge that chemical and enzymatic labeling techniques are typically less than 100 percent efficient. In some embodiments, “substantially all” means at least 80 percent of all monomer have fluorescent labels attached. In other embodiments, “substantially all” means at least 90 percent of all monomer have fluorescent labels attached. In other embodiments, “substantially all” means at least 95 percent of all monomer have fluorescent labels attached. Mutually quenching sets of fluorescent dyes have the following properties: (i) each member quenches fluorescence of every member (for example, by FRET or by static or contact mechanisms), and (ii) each member generates a distinct fluorescent signal when excited and when in a non-quenched state. That is, if a mutually quenching set consists of two dyes, D1 and D2, then (i) D1 is self-quenched (e.g. by contact quenching with another D1 molecule) and it is quenched by D2 (e.g. by contact quenching) and (ii) D2 is self-quenched (e.g. by contact quenching with another D2 molecule) and it is quenched by D1 (e.g. by contact quenching). Guidance for selecting fluorescent dyes or labels for mutually quenching sets may be found in the following references, which are incorporated herein by reference: Johansson, Methods in Molecular Biology, 335: 17-29 (2006); Marras et al, Nucleic Acids Research, 30: e122 (2002); and the like. In some embodiments, members of a mutually quenching set comprise organic fluorescent dyes that components or moieties capable of stacking interactions, such as aromatic ring structures. Exemplary mutually quenching sets of fluorescent dyes, or labels, may be selected from rhodamine dyes, fluorescein dyes and cyanine dyes. In one embodiment, a mutually quenching set may comprise the rhodamine dye, TAMRA, and the fluorescein dye, FAM. In another embodiment, mutually quenching sets of fluorescent dyes may be formed by selecting two or more dyes from the group consisting of Oregon Green 488, Fluorescein-EX, fluorescein isothiocyanate, Rhodamine Red-X, Lissamine rhodamine B, Calcein, Fluorescein, Rhodamine, one or more BODIPY dyes, Texas Red, Oregon Green 514, and one or more Alexa Fluors. Respresentative BODIPY dyes include BODIPY FL, BODIPY R6G, BODIPY TMR, BODIPY 581/591, BODIPY TR, BODIPY 630/650 and BODIPY 650/665. Representative Alexa Fluors include Alexa Fluor 350, 405, 430, 488, 500, 514, 532, 546, 555, 568, 594, 610, 633, 635, 647, 660, 680, 700, 750 and 790.

As above, in some embodiments, a monomer sequence of a target polynucleotide is determined by carrying out separate reactions (one for each kind of monomer) in which copies of the target polynucleotide have each different kind of monomer labeled with a mutually- or self-quenching fluorescent label. In other embodiments, a monomer sequence of a target polynucleotide is determined by carrying out separate reactions (one for each kind of monomer) in which copies of the target polynucleotide have each different kind of monomer labeled with a different mutually quenching fluorescent label selected from the same mutually quenching set. In embodiments in which a mutually quenching set contains only two dyes, then a selected monomer (say, monomer X) is labeled with a first mutually quenching dye and every other kind of monomer (i.e., not-monomer X) is labeled with a second mutually quenching dye from the same set. Thus, steps of the embodiment generate a sequence of two different fluorescent signals, one indicating monomer X and another indicating not-monomer X.

In some embodiments, a single fluorescent label (for example, attached to a single kind of monomer in a polynucleotide comprising multiple kinds of monomers) may be used that is self-quenching when attached to adjacent monomers (of the same kind) on a polynucleotide, such as adjacent nucleotides of a polynucleotide. Exemplary self-quenching fluorescent labels include, but are not limited to, Oregon Green 488, fluorescein-EX, FITC, Rhodamine Red-X, Lissamine rhodamine B, calcein, fluorescein, rhodamine, BODIPYS, and Texas Red, e.g. which are disclosed in Molecular Probes Handbook, 11th Edition (2010).

### Embodiments Employing Quenching Agents

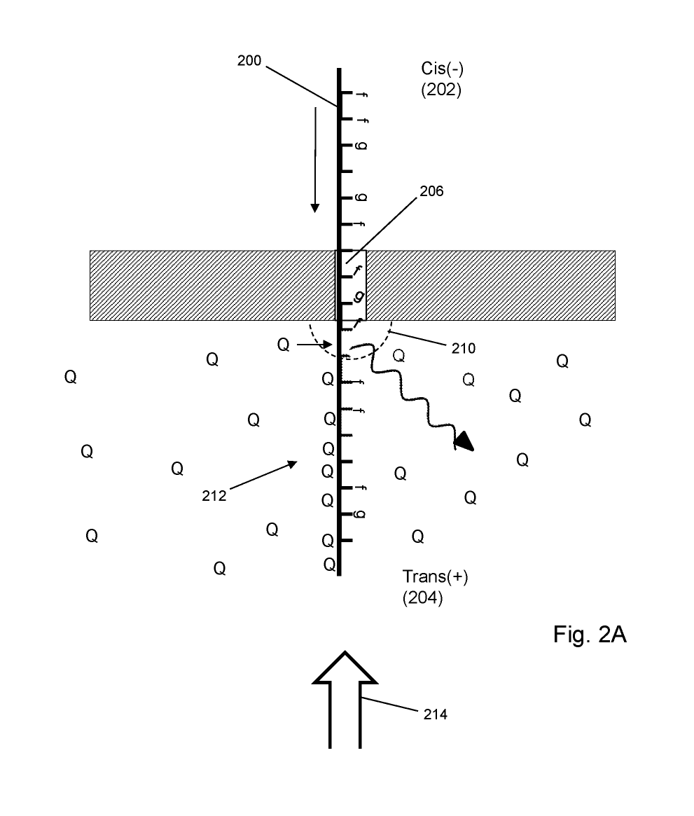

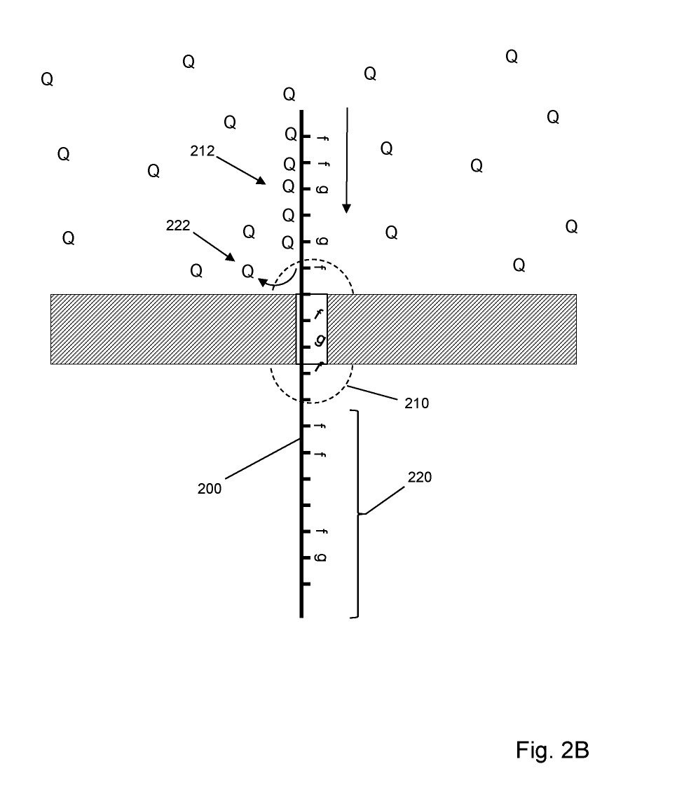

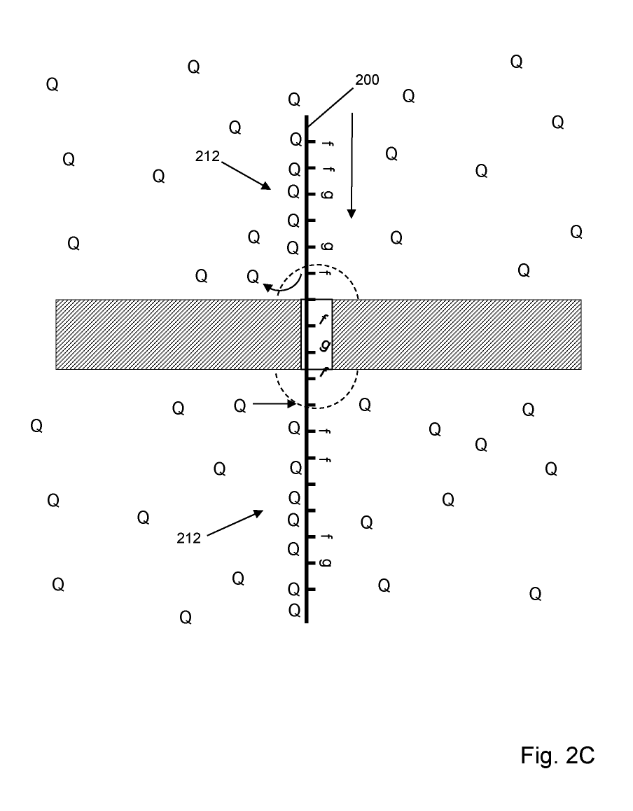

FIGS. 2A-2C illustrate different embodiments corresponding to where quenching agents are applied in a nanopore device: trans chamber only (FIG. 2A), cis chamber only (FIG. 2B), or both cis and trans chambers (FIG. 2C). In FIG. 2A, labeled polynucleotide (200) is illustrated translocating nanopore (206) of solid phase membrane (208) from cis chamber (202) to trans chamber (204). Immersed in trans chamber (204) are non-fluorescent quenching agents (205) designated by “Q”. Quenching agents of the invention are soluble under translocation conditions for labeled polynucleotide (200), and under the same conditions, quenching agents bind to single stranded polynucleotides, such as (200), without substantial sequence specificity. As explained more fully below, a large variety of non-fluorescent quenching agents are available for use with the invention, which include derivatives of many well-known organic dyes, such as asymmetric cyanine dyes, as well as conjugates of such compounds and oligonucleotides and/or analogs thereof. In this embodiment, selection of the type and concentration of quenching agent and the translocation speed define detection zone (210). In some embodiments, “detection zone” means a region or volume (which may be contiguous or non-contiguous) from which fluorescent signals are collected to form the raw data from which information, such as sequence information, about a labeled polynucleotide is determined. Fluorescent labels in trans chamber (204) outside of detection zone (210) are substantially quenched by quenching agents (205) bound to the portion of labeled polynucleotide (200) in trans chamber (204). In some embodiments, quenching agents comprise an oligonucleotide or analog conjugated to one or more quenching moieties based on organic dyes as described more fully below. Embodiments of FIG. 2A may be employed when, for example, solid phase membrane (208) is or comprises an opaque layer so that fluorescent labels in cis chamber (202) are substantially non-excited.

FIG. 2B shows substantially the same elements as those in FIG. 2A with the exception that quenching agents (205) are disposed in cis chamber (202). This configuration may be desirable under circumstances where undesired evanescent waves, or like non-radiative light energy, extend to cis chamber (202) and excite fluorescent labels which generate fluorescent signals that are collected. Quenching agents (205) that bind to labeled polynucleotide (200) in cis chamber (202) reduce or eliminate such fluorescent signals. In some embodiments, quenching agents (205) and cross-section of nanopore (206) are selected so that quenching agents (205) are excluded from translocating through nanopore (206). In some embodiments, this may be achieved by using protein nanopore α-hemolysin and quenching agents comprising conjugates of oligonucleotides or analogs thereof and one or more quenching compounds, as described more fully below.

FIG. 2C illustrates an embodiment where quenching agents (205) are present in both cis chamber (202) and trans chamber (204), which provides the advantages described for the embodiments of both FIGS. 2A and 2B.

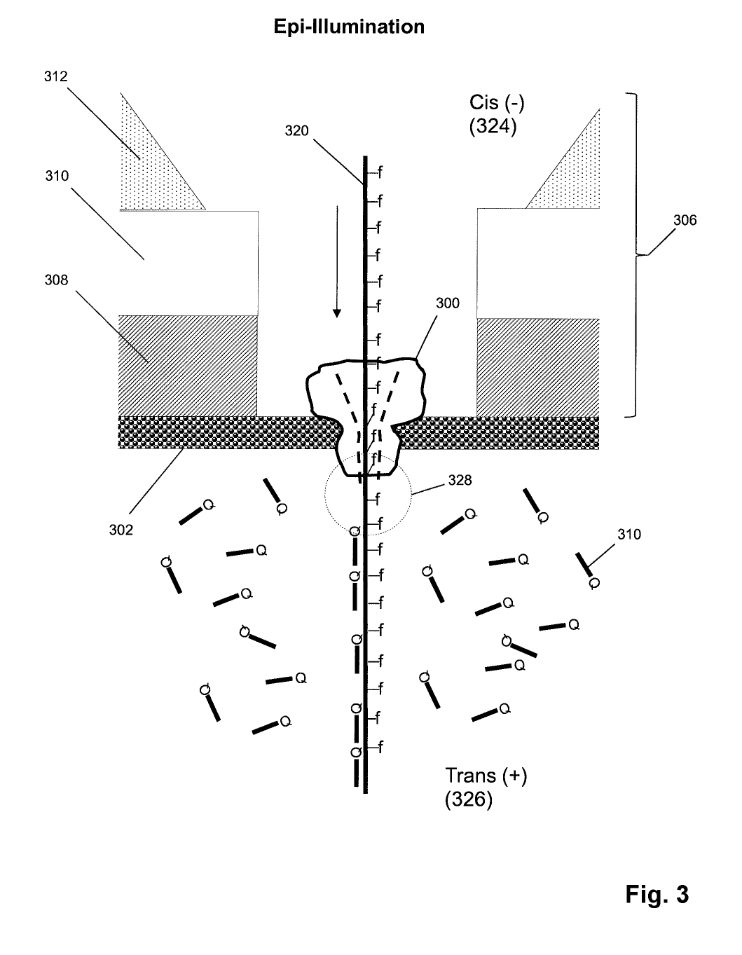

FIG. 3 illustrates an embodiment which includes the following elements: protein nanopore (300) disposed in a central opening of annular DNA sheet (302); epi-illumination of fluorescent labels with opaque layer (308) in solid phase membrane (306) to prevent or reduce background fluorescence; and quenching agents (310) disposed in trans chamber (326). As above, polynucleotide (320) with fluorescently labeled nucleotides (labels being indicated by “f”, as with (322)) is translocated through nanopore (300) from cis chamber (324) to trans chamber (326). Oligonucleotide quenchers (310) are disposed in trans chamber (326) under conditions (e.g. concentration, temperature, salt concentration, and the like) that permits hybridization of oligonucleotide quenchers (328) to portions of polynucleotide (320) emerging from nanopore (300). Nanopore (300) may be selected so that signals from fluorescent labels are suppressed during transit of the nanopore as described in Huber et al, U.S. patent publication US 2016/0076091, which is incorporated herein by reference. Thus, when labeled nucleotides emerge from nanopore (300) in region (328) they become unsuppressed and capable of generating a signal. With most if not all forms of direct illumination (e.g. non-FRET) such emerged labels would continue to emit fluorescence as they travel further into trans chamber (326), thereby contributing greatly to a collected signal. With quenching agents in trans chamber (326) that bind to the emerging polynucleotide, such emissions can be significantly reduced and can define detection zone (328) from which collected signals can be analyzed to give nucleotide sequence information about polynucleotide (320). In some embodiments, a fluorescent signal from a single fluorescent label is detected from detection zone (328) during a detection period as the labeled polynucleotide moves through the detection zone. In other embodiments, a plurality of fluorescent signals is collected from a plurality of fluorescent labels in detection zone (328) during a predetermined time period. In some embodiments, such detection period is less than 1 msec, or less than 0.1 msec, or less than 0.01 msec. In some embodiments, such detection period is at least 0.01 msec, or at least 0.1 msec, or at least 0.5 msec.

Quenching agents of the invention comprise any compound (or set of compounds) that under nanopore sequencing conditions is (i) substantially non-fluorescent, (ii) binds to single stranded nucleic acids, particularly single stranded DNA, and (iii) absorbs excitation energy from other molecules non-radiatively and releases it non-radiatively. In some embodiments, quenching agents further bind non-covalently to single stranded DNA. A large variety of quenching compounds are available for use with the invention including, but not limited to, non-fluorescent derivatives of common synthetic dyes such as cyanine and xanthene dyes, as described more fully below. Guidance in selecting quenching compounds may be found in U.S. Pat. Nos. 6,323,337; 6,750,024 and like references, which are incorporated herein by reference.

In some embodiments, a quenching agent may be a single stranded DNA binding dye that has been covalently modified with a heavy atom that is known to quench fluorescence (such as bromine or iodine), or covalently modified with other groups known to quench fluorescence, such as a nitro group or a azo group. An example of dye that is known to bind single stranded DNA is Sybr Green (Zipper et al, (2004), Nucleic Acids Research. 32 (12)). Incorporation of a nitro, bromine, iodine, and/or azo groups into the cynanine Sybr Green structure provides a single stranded DNA binding group moiety that will quench fluorescent labels that might be present on a DNA.

In some embodiments, quenching agents comprise a binding moiety and one or more quenching moieties. Binding moieties may include any compound that binds to single stranded nucleic acids without substantial sequence specificity. Binding moieties may comprise peptides or oligonucleotides or analogs of either having modified linkages and/or monomers. Oligonucleotides and their analogs may provide binding to polynucleotides via duplex formation or via non-base paired aptameric binding. In some embodiments, binding moieties comprise an oligonucleotide or analog thereof having a length in the range of from 6 to 60 nucleotides. Such oligonucleotides or analogs may be conjugated to one quenching moiety or to a plurality of quenching moieties. In some embodiments, the plurality of quenching moieties conjugated to each oligonucleotide or analog is 2 or 3. Quenching moieties conjugated to a binding moiety may be the same or different. In some embodiments, whenever a binding moiety is an oligonucleotide or analog, two quenching moieties are conjugated thereto, one at a 5′ end and one at a 3′ end of the oligonucleotide. Oligonucleotides or analogs having from 2 to 3 quenching moieties may be synthesized using conventional linkage and synthetic chemistries, for example, as disclosed in the references cited herein.

Oligonucleotides or analogs may be provided as a single species or they may be provided as mixtures of a plurality of oligonucleotides or analogs with different sequences, and therefore, different binding specificities. In some embodiments, oligonucleotides or analogs are random sequence polymers; that is, they are provided as mixtures of every possible sequence of a given length. For example, such oligonucleotides or analogs may be represented by the formulas, “NNNNNN” for 6-mers, or “NNNNNNNN” for 8-mers, wherein N may be A, C, G or T, or an analog thereof.

“Analogs” in reference to oligonucleotides means an oligonucleotide that contains one or more nucleotide analogs. As described in the definition section, a “nucleotide analog” is a nucleotide that may have a modified linkage moiety, sugar moiety or base moiety. Exemplary oligonucleotide analogs that may be used with the invention include, but are not limited to, peptide nucleic acids (PNAs), locked nucleic acids (LNAs) (2′-O-methyl RNA), phosphorothioate oligonucleotides, bridged nucleic acids (BNAs), or the like.

In some embodiments, oligonucleotide binding moieties comprise universal bases; that is, they contain one or more nucleotide analogs that can replace any of the four natural nucleotides without destabilizing base-pair interactions. Nucleotide analogs having universal base properties are described in Loakes, Nucleic Acids Research, 29(12): 2437-2447 (2001), which is incorporated herein by reference. In some embodiments, oligonucleotide binding moieties comprise 2′-deoxyinosine, 7-deaza-2′-deoxyinosine, 2-aza-2′-deoxyinosine, 3-nitropyrrole nucleotides, 5-nitroindole nucleotides, or the like.

In some embodiments, quenching agents may comprise a combination of two or more compounds that act together to quench undesired fluorescent signals of a single stranded labeled polynucleotide. For example, a quenching agent may comprise an oligonucleotide (e.g., polydeoxyinosine) that may form a duplex with the labeled polynucleotide and separately a double stranded intercalator that is a quencher. Thus, whenever the polydeoxyinosine binds to a labeled polynucleotide, the quenching intercalator binds to the resulting duplex and quenches fluorescent signals from the polynucleotide.

Any synthetic dye that can detectably quench fluorescent signals of the fluorescent labels of a labeled polynucleotide is an acceptable quenching moiety for the purposes of the invention. Specifically, as used in the invention, the quenching moieties possess an absorption band that exhibits at least some spectral overlap with an emission band of the fluorescent labels on a labeled polynucleotide. This overlap may occur with emission of the fluorescent label (donor) occurring at a lower or even higher wavelength emission maximum than the maximal absorbance wavelength of the quenching moiety (acceptor), provided that sufficient spectral overlap exists. Energy transfer may also occur through transfer of emission of the donor to higher electronic states of the acceptor. One of ordinary skill in the art determines the utility of a given quenching moiety by examination of that dye's excitation bands with respect to the emission spectrum of the fluorescent labels being used.

Typically, fluorescence quenching in the invention occurs through Fluorescence Resonance Energy Transfer (FRET or through the formation of charge transfer complexes) between a fluorescent label and a quenching moiety of the invention. The spectral and electronic properties of the donor and acceptor compounds have a strong effect on the degree of energy transfer observed, as does the separation distance between the fluorescent labels on the labeled polynucleotide and the quenching moiety. As the separation distance increases, the degree of fluorescence quenching decreases.

A quenching moiety may be optionally fluorescent, provided that the maximal emission wavelength of the dye is well separated from the maximal emission wavelength of the fluorescent labels when bound to labeled polynucleotides. Preferably, however, the quenching moiety is only dimly fluorescent, or is substantially non-fluorescent, when covalently conjugated to a oligonucleotide or analog. Substantially non-fluorescent, as used herein, indicates that the fluorescence efficiency of the quenching moiety in an assay solution as described for any of the methods herein is less than or equal to 5 percent, preferably less than or equal to 1 percent. In other embodiments, the covalently bound quenching moiety exhibits a quantum yield of less than about 0.1, more preferably less than about 0.01. In some embodiments, the fluorescence of fluorescent labels associated with a quenching oligonucleotide of the invention is quenched more than 50% relative to the same oligonucleotide associated with the same fluorescent labels in the absence of the covalently bound quenching moiety. In another embodiment, the fluorescent labels are quenched more than 90% relative to the unlabeled oligonucleotide. In yet another embodiment, the nucleic acid stains are quenched more than 95% relative to the unlabeled oligonucleotide.

In some embodiments, a quenching moiety may be a pyrene, an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine, a carbocyanine, a carbostyryl, a porphyrin, a salicylate, an anthranilate, an azulene, a perylene, a pyridine, a quinoline, a coumarin (including hydroxycoumarins and aminocoumarins and fluorinated and sulfonated derivatives thereof (as described in U.S. Pat. No. 5,830,912 to Gee et al. (1998) and U.S. Pat. No. 5,696,157 to Wang et al. (1997), incorporated by reference), a polyazaindacene (e.g. U.S. Pat. No. 4,774,339 to Haugland, et al. (1988); U.S. Pat. No. 5,187,288 to Kang, et al. (1993); U.S. Pat. No. 5,248,782 to Haugland, et al. (1993); U.S. Pat. No. 5,274,113 to Kang, et al. (1993); U.S. Pat. No. 5,433,896 to Kang, et al. (1995); U.S. Pat. No. 6,005,113 to Wu et al. (1999), all incorporated by reference), a xanthene, an oxazine or a benzoxazine, a carbazine (U.S. Pat. No. 4,810,636 to Corey (1989), incorporated by reference), or a phenalenone or benzphenalenone (U.S. Pat. No. 4,812,409 Babb et al. (1989), incorporated by reference).

In other embodiments, quenching moieties that are substantially non-fluorescent dyes include in particular azo dyes (such as DABCYL or DABSYL dyes and their structural analogs), triarylmethane dyes such as malachite green or phenol red, 4′,5z-diether substituted fluoresceins (U.S. Pat. No. 4,318,846 (1982)), or asymmetric cyanine dye quenchers (PCT Int. App. WO 99 37,717 (1999)).

In embodiments where the quenching moiety is a xanthene, the synthetic dye is optionally a fluorescein, a rhodol (U.S. Pat. No. 5,227,487 to Haugland, et al. (1993), incorporated by reference), or a rhodamine. As used herein, fluorescein includes benzo- or dibenzofluoresceins, seminaphthofluoresceins, or naphthofluoresceins. Similarly, as used herein rhodol includes seminaphthorhodafluors (U.S. Pat. No. 4,945,171 to Haugland, et al. (1990), incorporated by reference). Xanthenes include fluorinated derivatives of xanthene dyes (Int. Publ. No. WO 97/39064, Molecular Probes, Inc. (1997), incorporated by reference), and sulfonated derivatives of xanthene dyes (Int. Publ. No. WO 99/15517, Molecular Probes, Inc. (1999), incorporated by reference). As used herein, oxazines include resorufms, aminooxazinones, diaminooxazines, and their benzo-substituted analogs.

In further embodiments, the quenching moiety is an substantially nonfluorescent derivative of 3- and/or 6-amino xanthene that is substituted at one or more amino nitrogen atoms by an aromatic or heteroaromatic ring system, e.g. as described in U.S. Pat. No. 6,399,392, which is incorporated herein by reference. These quenching dyes typically have absorption maxima above 530 nm, have little or no observable fluorescence and efficiently quench a broad spectrum of luminescent emission, such as is emitted by chemilumiphores, phosphors, or fluorophores. In one embodiment, the quenching dye is a substituted rhodamine. In another embodiment, the quenching compound is a substituted rhodol.

In still other embodiments, a quenching moiety may comprise one or more non-fluorescent quenchers known as Black Hole Quenchers™ compounds (BHQs) described in the following patents, which are incorporated herein by reference: U.S. Pat. No. 7,019,129; 7,109,312; 7,582,432; 8,410,025; 8,440,399; 8,633,307; 8,946,404; 9,018,369; or 9,139,610.

Additional quenching moieties are disclosed in the following, which are incorporated herein by reference: U.S. Pat. Nos. 6,699,975; 6,790,945; and 8,114,979.

### Embodiments Employing Two or Three Optical Labels

In some embodiments, as few as two different kinds of nucleotide are labeled with different optical labels that generate distinguishable optical signals for the selected kinds of nucleotide in both sense strands and antisense strands of target polynucleotides. For example, C's and T's of the complementary strands of each target polynucleotide may be replaced by labeled analogs, wherein the labels of the C and T analogs are capable of generating distinct optical signals. Optical signatures are then generated by translocating the labeled strands through nanopores where nucleotides of the strands are constrained to pass sequentially through an optical detection region where their labels are caused to generate optical signals. In some embodiments, information from optical signatures from both sense and antisense strands are combined to determine a nucleotide sequence of target polynucleotides.

In some embodiments, the selected kinds of nucleotides of target polynucleotides are replaced by labeled nucleotide analogs in an extension reaction using a nucleic acid polymerase. Labeled strands of target polynucleotides are translocated through nanopores that constrain the nucleotides of strands to move single file through an optical detection region where they are excited so that they produce an optical signal. A collection of optical signals for an individual strand is referred to herein as an optical signature of the strand. In some embodiments, where a strand and its complement (i.e. sense and antisense strands) are linked, for example, via a hairpin adaptor, a single optical signature may include optical signals from optical labels on nucleotides from both the sense strand and the antisense strand. In other embodiments, different strands of a target polynucleotide may separately generate two different optical signatures which may be combined, or used together, for analysis, as mentioned above. Such separately analyzed strands may be associated after generation of optical signatures, for example, by using molecular tags (which may be, for example, oligonucleotide segments attached to target polynucleotides in a known position, length and sequence pattern and diversity to permit ready association). As noted below, optical signature of the invention may comprise mixed optical signals in that the signal detected in each detection interval may comprise contributions from multiple optical labels emitting within a resolution limited area or volume; that is, they may (for example) be mixed FRET signals, as described by Huber et al, U.S. patent publication US20160076091, which is incorporated herein by reference.

As mentioned above, in some embodiments, methods of the invention may be implemented with the following steps: (a) copying a strand of a double stranded polynucleotide so that nucleotide analogs with distinct optical labels are substituted for at least two kinds of nucleotide to form a labeled strand; (b) copying a complement of the strand so that said nucleotide analogs are substituted for the same at least two kinds of nucleotide to form a labeled complement; (c) translocating the labeled stand through a nanopore so that the nucleotides of the labeled strand pass single file through a detection zone where optical labels are excited to generate optical signals; (d) quenching fluorescent signals from excited fluorescent labels outside of the detection zone; (e) detecting a time series of optical signals from the optical labels as the labeled strand translocates through the nanopore to produce a strand optical signature; (f) translocating the labeled complement through a nanopore so that the nucleotides of the labeled complement pass single file through an excitation zone where optical labels are excited to generate optical signals; (g) quenching fluorescent signals from excited fluorescent labels outside of the detection zone; (h) detecting a time series of optical signals from the optical labels as the labeled complement translocates through the nanopore to produce a complement optical signature; (i) determining a sequence of the double stranded polynucleotide from the strand optical signature and the complement optical signature. In some embodiments, two kinds of nucleotide are labeled, which may be C's and T's, C's and G's, C's and A's, T's and G's, T's and A's, or G's and A's. In some embodiments, pyrimidine nucleotides are labeled. In other embodiments, purine nucleotides are labeled. In some embodiments, selected kinds of nucleotides of a strand are labeled by incorporating labeled analog dNTPs of the selected kind of nucleotides in a primer extension reaction using a nucleic acid polymerase. In other embodiments, selected kinds of nucleotides of a strand are labeled by incorporating analog dNTPs of the selected kinds of nucleotides in an extension reaction, wherein the analog dNTPs are derivatized with orthogonally reactive functionalities that allow attachment of different labels to different kinds of nucleotides in a subsequent reaction. This latter labeling approach is disclosed in Jett et al, U.S. Pat. No. 5,405,747, which is incorporated herein by reference.

In some embodiments, three kinds of nucleotide are labeled, which may include labeling C's with a first optical label, T's with a second optical label, and G's and A's with a third optical label. In other embodiments, the following groups of nucleotides may be labeled as indicated: C's and G's with a first optical label and second optical label, respectively, and T's and A's with a third optical label; C's and A's with a first optical label and second optical label, respectively, and T's and G's with a third optical label; T's and G's with a first optical label and second optical label, respectively, and C's and A's with a third optical label; A's and G's with a first optical label and second optical label, respectively, and T's and C's with a third optical label.

In some embodiments, optical labels are fluorescent acceptor molecules that generate a fluorescent resonance energy transfer (FRET) signal after energy transfer from a donor associated with a nanopore. In some embodiments, as described further below, donors may be optically active nanoparticles, such as, quantum dots, nanodiamonds, or the like. Selection of particular combinatins of acceptor molecules and donors are design choices for one of ordinary skill in the art. In some embodiments, some of which are described more fully below, a single quantum dot is attached to a nanopore and is excited to fluoresce using an excitation beam whose wavelength is sufficiently separated, usually lower (i.e. bluer), so that it does not contribute to FRET signals generated by acceptors. Likewise, a quantum dot is selected whose emission wavelength overlaps the absorption bands of both acceptor molecules to facilitate FRET interactions. In some embodiments, two donors may be used for each excitation zone of a nanopore, wherein the emission wavelength of each is selected to optimally overlap the absorption band of a different one of the acceptor molecules.

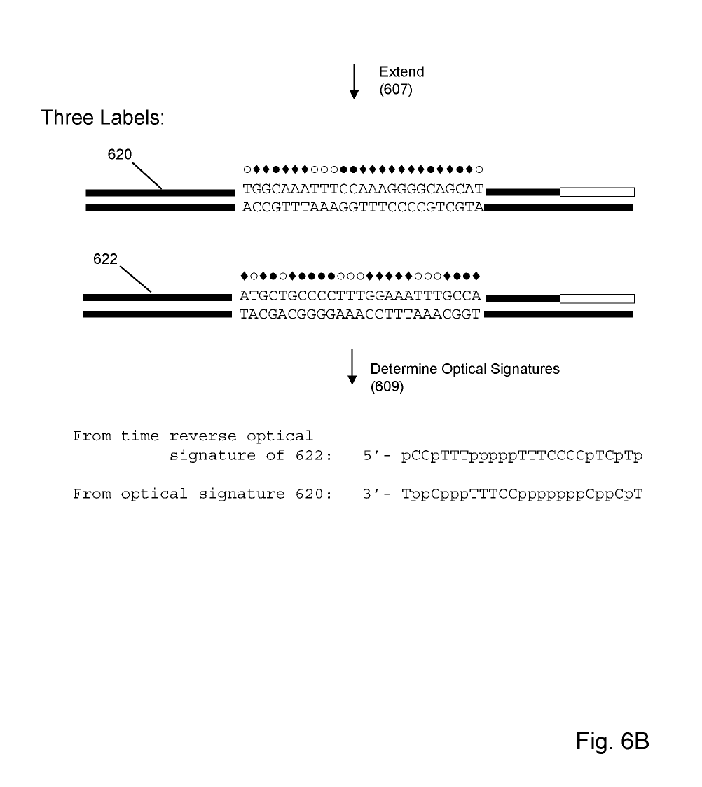

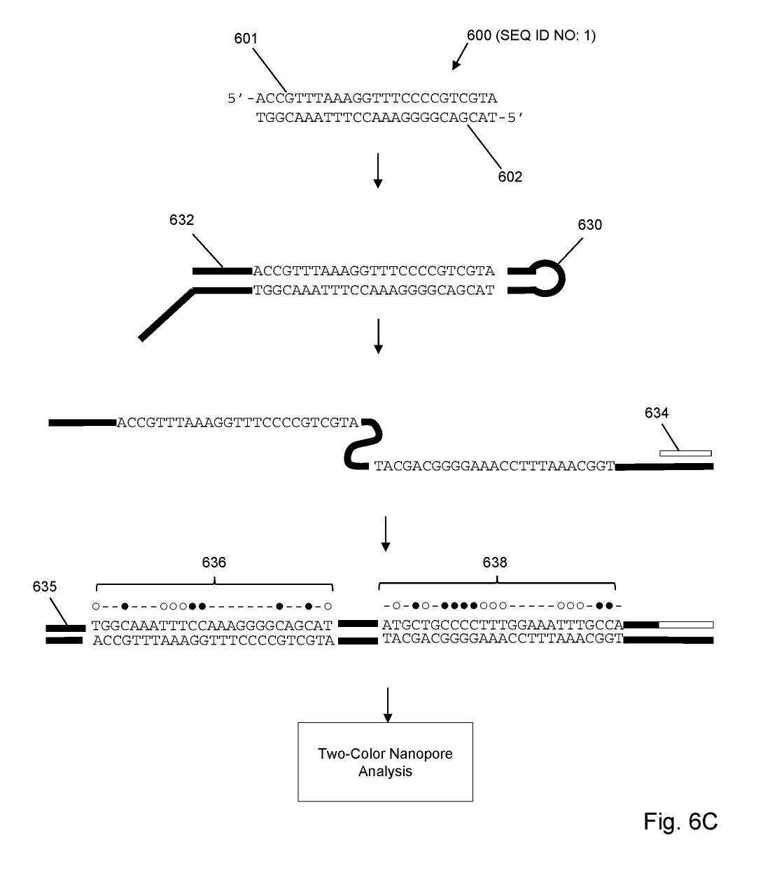

In FIG. 6A, double stranded target polynucleotide (600) (SEQ ID NO: 1) consists of sense strand (601) and complementary antisense strand (602), to which is ligated (603) “Y” adaptors (604) and (606) using conventional methods, e.g. Weissman et al, U.S. Pat. No. 6,287,825; Schmitt et al, U.S. patent publication US2015/004468; which are incorporated herein by reference. Arms (608) and (610) of adaptors (604 and 606, respectively) include primer binding sites to which primers (616) and (618) are annealed (605). Double stranded portions (612) and (614) may include tag sequences, e.g. one or both may include randomers of predetermined length and composition, which may be used for later re-association of the strands, for example, to obtain sequence information from the respective optical signatures of the strands. After annealing primers (616) and (618), they may be extended (607) by a nucleic acid polymerase in the presence of (for example, as illustrated) labeled dUTP analogs (labels shown as open circles in the incorporated nucleotides) and labeled dCTP analogs (labels shown as filled circles in the incorporated nucleotides) and natural unlabeled dGTPs and dATPs (with neither unlabeled dTTP nor unlabeled dCTP being present so that the analogs are fully substituted in the extended strands). The absence of labels on G's and A's are illustrated as dashes above the incorporated nucleotides. In an ideal detection system without noise, the sequence of open circles, filled circles and dashes would be good representations of optical signatures generated by the indicated sense and antisense strands as they pass through an excitation zone of a nanopore.

In FIG. 6B, extension products (620) and (622) are illustrated for an alternative embodiment employing three labels. Incorporated labeled dUTP analogs are shown as open circles and incorporated labeled dCTP analogs are shown as filled circles, as above. Incorporated labeled dATP and dGTP analogs are shown as filled diamonds. FIG. 6C illustrates an embodiment in which two labels are used and sense and antisense strands are linked by means of hairpin adaptor (630), for example, as taught in U.S. patent publications US 2015/0152492 and US 2012/0058468, which are incorporated herein by reference. Tailed adaptor (632) and hairpin adaptor (630) are ligated to target polynucleotide (600) (SEQ ID NO: 1). After denaturation and annealing of primer (634), an extension reaction produces extension product (635) which includes segment (636), the labeled complement of strand (601) and segment (638), the labeled reverse complement of strand (601). After translocation of extension product (635) through a nanopore and generation of an optical signature the sequence of target polynucleotide (600) (SEQ ID NO: 1) can be determined. Optionally, the sequence of hairpin (630) may be selected so that a predetermined pattern of labels is incorporated during the extension reaction, which may be used to assist in the analysis of the optical signature, e.g. by indicating where segment (636) ends and where segment (638) begins, or the like.

Guidance in selecting the kinds of nucleotide to label, kinds of labels and linkers for attaching them to bases, and nucleic acid polymerases for extension reactions in the presence of dNTP analogs can be found in the following references, which are incorporated by reference: Goodman et al, U.S. Pat. No. 5,945,312; Jett et al, U.S. Pat. No. 5,405,747; Muehlegger et al, U.S. patent publication US2004/0214221; Giller et al, Nucleic Acids Research, 31(10): 2630-2635 (2003); Tasara et al, Nucleic Acids Research, 31(10): 2636-2646 (2003); Augustin et al, J. Biotechnology, 86: 289-301 (2001); Brakmann, Current Pharmacuetical Biotechnology, 5(1): 119-126 (2004); and the like. Exemplary nucleic acid polymerases for use with the invention include, but are not limited to, Vent exo, Taq, E. coli Pol I, Tgo exo⁺, Klenow fragment exo, Deep Vent exo, and the like. In some embodiments, exemplary nucleic acid polymerases include, but are not limited to, Vent exo and Klenow fragment exo⁺. Exemplary fluorescent labels for dNTP analogs include, but are not limited to, Alexa 488, AMCA, Atto 655, Cy3, Cy5, Evoblue 30, fluorescein, Gnothis blue 1, Gnothis blue 2, Gnothis blue 3, Dy630, Dy635, MR121, rhodamine, Rhodamine Green, Oregon Green, TAMRA, and the like. Exemplary fluorescent labels for dUTP analogs include, but are not limited to, Alexa 488, AMCA, Atto 655, Cy3, Cy5, Dy630, Dy665, Evoblue 30, Evoblue 90, fluorescein, Gnothis blue 1, Gnothis blue 2, Gnothis blue 3, MR121, Oregon Green, rhodamine, Rhodamine Green, TAMRA, and the like. Exemplary fluorescent labels for dCTP analogs include, but are not limited to, Atto 655, Cy5, Evoblue 30, Gnothis blue 3, rhodamine, Rhodamine Green, TAMRA, and the like. Exemplary fluorescent labels for dATP analogs include, but are not limited to, Atto 655, Cy5, Evoblue 30, Gnothis blue 3, Rhodamine Green, and the like. Exemplary fluorescent labels for dGTP analogs include, but are not limited to, Evoblue 30, Gnothis blue 3, Rhodamine Green, and the like. Exemplary pairs of fluorescent labels for dUTP analogs and dCTP analogs include, but are not limited to, (TAMRA, Rhodamine Green), (Atto 655, Evoblue 30), (Evoblue 30, Atto 655), (Evoblue 30, Gnothis blue 3), (Evoblue 30, Rhodamine Green), (Gnothis blue 1, Rhodamine Green), (Gnothis blue 2, Atto 655), Gnothis blue 3, Cy5), and the like.

FIG. 6C illustrates an embodiment in which two labels are used and sense and antisense strands are linked by means of hairpin adaptor (630), for example, as taught in U.S. patent publications US 2015/0152492 and US 2012/0058468, which are incorporated herein by reference. Tailed adaptor (632) and hairpin adaptor (630) are ligated to target polynucleotide (600). After denaturation and annealing of primer (634), an extension reaction produces extension product (635) which includes segment (636), the labeled complement of strand (601) and segment (638), the labeled reverse complement of strand (601). After translocation of extension product (635) through a nanopore and generation of an optical signature the sequence of target polynucleotide (600) can be determined. Optionally, the sequence of hairpin (630) may be selected so that a predetermined pattern of labels is incorporated during the extension reaction, which may be used to assist in the analysis of the optical signature, e.g. by indicating where segment (636) ends and where segment (638) begins, or the like.

### Optical Signal Detection

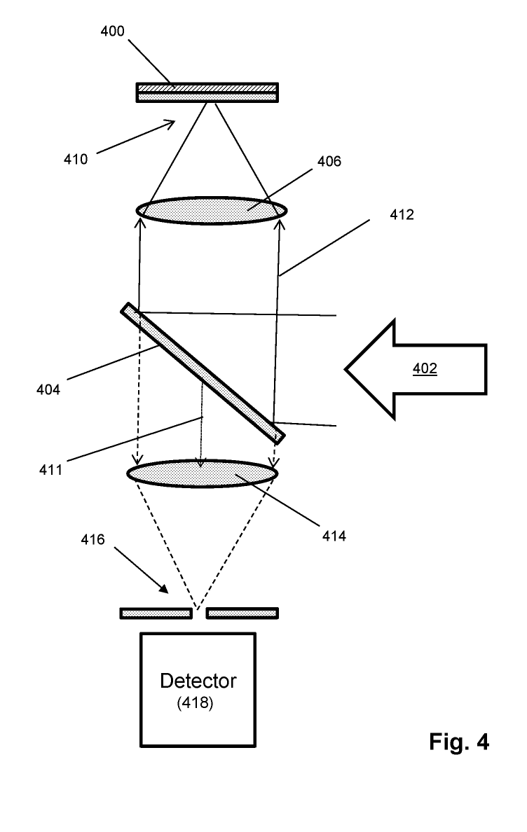

In some embodiments, an epi-illumination system, in which excitation beam delivery and optical signal collection occurs through a single objective, may be used for direct illumination of labels on a polymer analyte or donors on nanopores. The basic components of a confocal epi-illumination system for use with the invention is illustrated in FIG. 4. Excitation beam (402) is directed to dichroic (404) and onto (412) objective lens (406) which focuses (410) excitation beam (402) onto layered membrane (400), in which labels are excited directly to emit an optical signal, such as a fluorescent signal, or are excited indirectly via a FRET interaction to emit an optical signal. Such optical signal is collected by objective lens (406) and directed to dichroic (404), which is selected so that it passes light of optical signal (411) but reflects light of excitation beam (402). Optical signal (411) passes through lens (414) which focuses it through pinhole (416) and onto detector (418). When optical signal (411) comprises fluorescent signals from multiple fluorescent labels further optical components, filters, beam splitters, monochromators, or the like, may be provided for further separating the different fluorescent signals from different fluorescent labels.

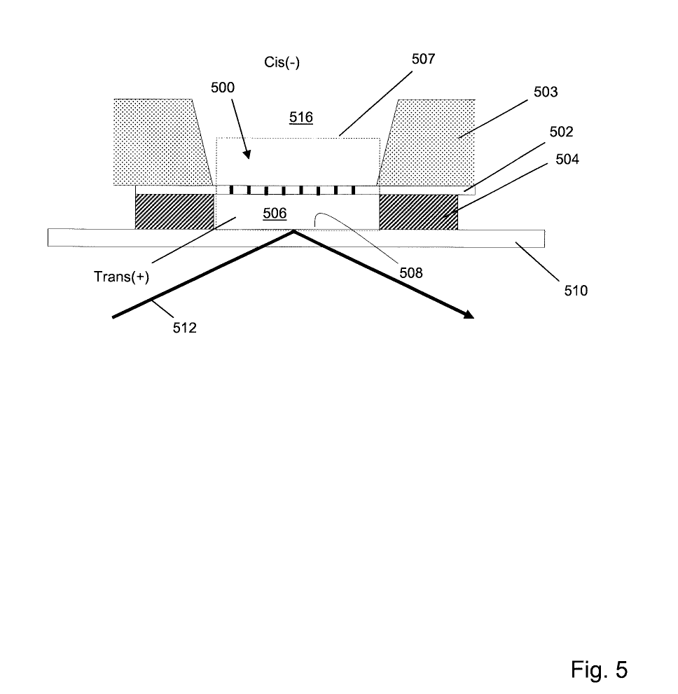

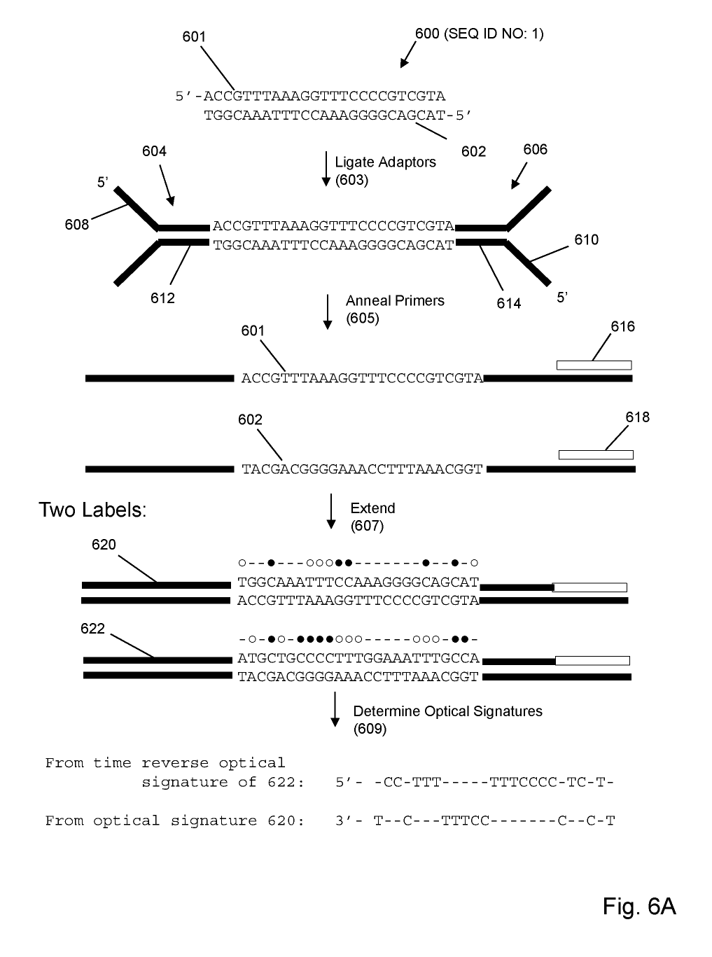

In some embodiments, labels on nucleotides may be excited by an evanescence field using an apparatus similar to that shown in FIG. 5, described in Soni et al, Review of Scientific Instruments, 81: 014301 (2010); and in U.S. patent publication 2012/0135410, which is incorporated herein by reference. In this apparatus, a very narrow second chamber on the trans side of a nanopore or nanopore array permits an evanescent field to extend from a surface of an underlying glass slide to establish detection zones both at entrances and exits of the nanopores, so that each optical measurement associated with a nanopore contains contributions from a plurality of labeled nucleotides. Array of apertures (500) (which may include protein nanopores inserted in a lipid bilayer), may be formed in silicon nitride layer (502), which may have a thickness in the range of from 20-100 nm. Silicon nitride layer (502) may be formed on a silicon support layer (503). Second chamber (506) may be formed by silicon nitride layer (502), silicon dioxide layer (504) which determines the height of second chamber (506), and surface (508) of glass slide (510). Silicon dioxide layer (504) may have a thickness in the range of from 50-100 nm. A desired evanescent field (507) extending from surface (508) across silicon nitride layer (502) may be established by directing light beam (512) at an appropriate angle relative to glass slide (510) so that TIR occurs. For driving labeled polynucleotide analytes through array (500), cis(−) conditions may be established in first chamber (516) and trans(+) conditions may be established in second chamber (506) with electrodes operationally connected to first and second chambers (506 and 521).

### Definitions

“Evanescent field” means a non-propagating electromagnetic field; that is, it is an electromagnetic field in which the average value of the Poynting vector is zero.

“FRET” or “Firster, or fluorescence, resonant energy transfer” means a non-radiative dipole-dipole energy transfer mechanism from an excited donor fluorophore to an acceptor fluorophore in a ground state. The rate of energy transfer in a FRET interaction depends on the extent of spectral overlap of the emission spectrum of the donor with the absorption spectrum of the acceptor, the quantum yield of the donor, the relative orientation of the donor and acceptor transition dipoles, and the distance between the donor and acceptor molecules, Lakowitz, Principles of Fluorescence Spectroscopy, Third Edition (Springer, 2006). FRET interactions of particular interest are those which result a portion of the energy being transferred to an acceptor, in turn, being emitted by the acceptor as a photon, with a frequency lower than that of the light exciting its donor (i.e. a “FRET signal”). “FRET distance” means a distance between a FRET donor and a FRET acceptor over which a FRET interaction can take place and a detectable FRET signal produced by the FRET acceptor.

“Kit” refers to any delivery system for delivering materials or reagents for carrying out a method of the invention. In the context of reaction assays, such delivery systems include systems that allow for the storage, transport, or delivery of reaction reagents (e.g., fluorescent labels, such as mutually quenching fluorescent labels, fluorescent label linking agents, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second or more containers contain mutually quenching fluorescent labels.

“Nanopore” means any opening positioned in a substrate that allows the passage of analytes through the substrate in a predetermined or discernable order, or in the case of polymer analytes, passage of their monomeric units through the substrate in a pretermined or discernible order. In the latter case, a predetermined or discernible order may be the primary sequence of monomeric units in the polymer. Examples of nanopores include proteinaceous or protein based nanopores, synthetic or solid state nanopores, and hybrid nanopores comprising a solid state nanopore having a protein nanopore immobilized therein either directly or indirectly as in the case of the present invention wherein an annular DNA sheet is employed as an adaptor between a solid state aperture and a protein nanopore. A nanopore may have an inner diameter of 1-10 nm or 1-5 nm or 1-3 nm. Examples of protein nanopores include but are not limited to, alpha-hemolysin, voltage-dependent mitochondrial porin (VDAC), OmpF, OmpC, MspA and LamB (maltoporin), e.g. disclosed in Rhee, M. et al., Trends in Biotechnology, 25(4) (2007): 174-181; Bayley et al (cited above); Gundlach et al, U.S. patent publication 2012/0055792; and the like, which are incorporated herein by reference. A synthetic nanopore, or solid-state nanopore, may be created in various forms of solid substrates, examples of which include but are not limited to silicones (e.g. Si3N4, SiO2), metals, metal oxides (e.g. Al2O3) plastics, glass, semiconductor material, and combinations thereof. A synthetic nanopore may be more stable than a biological protein pore positioned in a lipid bilayer membrane.

“Polymer” means a plurality of monomers connected into a linear chain. Usually, polymers comprise more than one type of monomer, for example, as a polynucleotide comprising A's, C's, G's and T's, or a polypeptide comprising more than one kind of amino acid. Monomers may include without limitation nucleosides and derivatives or analogs thereof and amino acids and derivatives and analogs thereof. In some embodiments, polymers are polynucleotides, whereby nucleoside monomers are connected by phosphodiester linkages, or analogs thereof.

“Polynucleotide” or “oligonucleotide” are used interchangeably and each mean a linear polymer of nucleotide monomers or analogs thereof. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moieties, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′-3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references. Likewise, the oligonucleotide and polynucleotide may refer to either a single stranded form or a double stranded form (i.e. duplexes of an oligonucleotide or polynucleotide and its respective complement). It will be clear to one of ordinary skill which form or whether both forms are intended from the context of the terms usage.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2nd Edition (Cold Spring Harbor Press, New York, 2003).

“Resolution limited area” is an area of a surface of a nanopore or nanowell array within which individual features or light emission sources cannot be distinguished by an optical signal detection system. Without intending to be limited by theory, such resolution limited area is determined by a resolution limit (also sometimes referred to as a “diffraction limit” or “diffraction barrier”) of an optical system. Such limit is determined by the wavelength of the emission source and the optical components and may be defined by d=X/NA, where d is the smallest feature that can be resolved, X is the wavelength of the light and NA is the numerical aperture of the objective lens used to focus the light. Thus, whenever two or more nanopores are within a resolution limited area and two or more optical signals are generated at the respective nanopores, an optical detection system cannot distinguish or determine which optical signals came from which nanopore. In accordance with the invention, a surface of a nanopore array may be partitioned, or subdivided, into non-overlapping regions, or substantially non-overlapping regions, corresponding to resolution limited areas. The size of such subdivisions corresponding to resolution limited areas may depend on a particular optical detection system employed. In some embodiments, whenever light emission sources are within the visible spectrum, a resolution limited area is in the range of from 300 nm² to 3.0 μm²; in other embodiments, a resolution limited area is in the range of from 1200 nm² to 0.7 μm²; in other embodiments, a resolution limited area is in the range of from 3×10⁴ nm² to 0.7 μm², wherein the foregoing ranges of areas are in reference to a surface of a nanopore or nanowell array. In some embodiments, the visible spectrum means wavelengths in the range of from about 380 nm to about 700 nm.

“Sequence determination”, “sequencing” or “determining a nucleotide sequence” or like terms in reference to polynucleotides includes determination of partial as well as full sequence information of the polynucleotide. That is, the terms include sequences of subsets of the full set of four natural nucleotides, A, C, G and T, such as, for example, a sequence of just A's and C's of a target polynucleotide. That is, the terms include the determination of the identities, ordering, and locations of one, two, three or all of the four types of nucleotides within a target polynucleotide. In some embodiments, the terms include the determination of the identities, ordering, and locations of two, three or all of the four types of nucleotides within a target polynucleotide. In some embodiments sequence determination may be accomplished by identifying the ordering and locations of a single type of nucleotide, e.g. cytosines, within the target polynucleotide “catcgc . . . ” so that its sequence is represented as a binary code, e.g. “100101 . . . ” representing “c-(not c)(not c)c-(not c)-c . . . ” and the like. In some embodiments, the terms may also include subsequences of a target polynucleotide that serve as a fingerprint for the target polynucleotide; that is, subsequences that uniquely identify a target polynucleotide, or a class of target polynucleotides, within a set of polynucleotides, e.g. all different RNA sequences expressed by a cell.

This disclosure is not intended to be limited to the scope of the particular forms set forth, but is intended to cover alternatives, modifications, and equivalents of the variations described herein. Further, the scope of the disclosure fully encompasses other variations that may become obvious to those skilled in the art in view of this disclosure. The scope of the present invention is limited only by the appended claims.

What is claimed is:

1. An article of manufacture for constraining movement of molecules, the article of manufacture comprising:
a solid state membrane having at least one aperture extending therethrough from a first side to a second side;
an annular DNA sheet having a central opening disposed on the first side of the solid state membrane such that the annular DNA sheet spans an aperture and the central opening is aligned with the aperture to provide fluid communication between the first side and the second side of the solid state membrane through the aperture; and
a protein nanopore immobilized in the central opening of the annular DNA sheet spanning the aperture.

2. The article of manufacture of claim 1 wherein said annular DNA sheet is bonded to said first side of said solid state membrane.

3. The article of manufacture of claim 1 wherein said molecules are constrained to move through said protein nanopore immobilized in said central opening.

4. The article of manufacture of claim 3 wherein said molecules are polynucleotides.

5. The article of manufacture of claim 1 wherein said protein nanopore is immobilized in said central opening by chemical cross-linking.

6. An article of manufacture for constraining movement of molecules, the article of manufacture comprising:
an solid state membrane having one or more apertures, the solid state membrane separating a first chamber from a second chamber wherein the solid state membrane has a first surface forming a boundary of the first chamber and having a reactive moiety coated thereon and wherein each of the one or more apertures has a cross-sectional area;
an annular DNA sheet having a central opening and having complementary moieties on a surface thereof, the complementary moieties forming a covalent linkage with the reactive moieties that bonds the annual DNA sheet on an aperture such that the annular DNA sheet spans the cross-sectional area thereof and the central opening thereof is aligned with the aperture to provide fluid communication between the first chamber and the second chamber; and
a protein nanopore immobilized in the central aperture of the aperture-spanning annular DNA sheet.

7. The article of manufacture of claim 6 wherein said molecules are constrained to move through said protein nanopore immobilized in said central opening.

8. The article of manufacture of claim 3 wherein said molecules are polynucleotides.

9. The article of manufacture of claim 6 wherein said protein nanopore is immobilized in said central opening by chemical cross-linking.

10. A method of determining a nucleotide sequence of a polynucleotide, the method comprising the steps of:
translocating a polynucleotide through a nanopore, wherein different kinds of nucleotides of the polynucleotide are capable of generating distinguishable signals as the nanopore constrains the nucleotides to move single file through a detection zone, and wherein the nanopore comprises (i) a solid state membrane having at least one aperture extending therethrough from a first side to a second side, (ii) an annular DNA sheet having a central opening disposed on the first side of the solid state membrane such that the annular DNA sheet spans an aperture and the central opening is aligned with the aperture to provide fluid communication between the first side and the second side of the solid state membrane through the aperture, and (iii) a protein nanopore immobilized in the central opening of the annular DNA sheet spanning the aperture;
detecting signals from nucleotides as the nucleotides pass through the detection zone; and
determining a sequence of nucleotide from the detected signals.

11. The method of claim 10 wherein different kinds of nucleotides of said polynucleotide are labeled with different fluorescent labels that generate distinguishable fluorescent signals, and wherein the different fluorescent labels are excited and their fluorescent signals are detected as they pass through said detection zone.

12. The method of claim 11 wherein said polynucleotide is a double stranded polynucleotide and wherein said method further includes the steps of:
copying a strand of the double stranded polynucleotide so that nucleotide analogs with said different fluorescent labels are substituted for at least two kinds of nucleotide to form a labeled strand;
copying a complement of the strand so that said nucleotide analogs are substituted for the same at least two kinds of nucleotide to form a labeled complement;
translocating the labeled stand through said nanopore so that the nucleotides of the labeled strand pass single file through an excitation zone where fluorescent labels are excited to generate optical signals;
detecting a time series of optical signals from the optical labels as the labeled strand translocates through the nanopore to produce a strand optical signature;
translocating the labeled complement through said nanopore so that the nucleotides of the labeled complement pass single file through an excitation zone where fluorescent labels are excited to generate optical signals;
detecting a time series of optical signals from the fluorescent labels as the labeled complement translocates through the nanopore to produce a complement optical signature;
determining a sequence of the double stranded polynucleotide from the strand optical signature and the complement optical signature.

13. The method of claim 10 wherein (i) said solid state membrane separates a first chamber from a second chamber, (ii) said solid state membrane has a first surface forming a boundary of the first chamber and having a reactive moiety coated thereon and (iii) each of said one or more apertures of said solid state membrane has a cross-sectional area; and wherein said annular DNA sheet has complementary moieties on a surface thereof, the complementary moieties forming a covalent linkage with the reactive moieties that bonds said annual DNA sheet on an aperture such that said annular DNA sheet spans the cross-sectional area thereof and said central opening thereof is aligned with the aperture to provide fluid communication between the first chamber and the second chamber.

14. The method of claim 10 wherein said protein nanopore is immobilized in said central opening by chemical cross-linking.
